(19) United States
(12) Patent Application Publication
(10) Pub. No.: US 2026/0301943 A1
(43) Pub. Date: Oct. 1, 2026

(54) INDIVIDUALIZED MEDICAL MODELING

(71) Applicant: Gemini Corp., San Francisco, CA (US)

(72) Inventor: Neal Solomon, Oakland, CA (US)

(73) Assignee: Gemini Corp., San Francisco, CA (US)

(21) Appl. No.: 19/094,885

(22) Filed: Mar. 29, 2025

Related U.S. Application Data
(60) Provisional application No. 63/631,056, filed on Apr. 8, 2024.
(60) Provisional application No. 63/680,852, filed on Aug. 8, 2024.

(51) Int. Cl. G16H 50/20; G16H 10/40; G16H 10/60; G16H 20/10; G16H 40/20; G16H 50/30; G16H 50/70

(52) U.S. Cl. CPC G16H 50/20; G16H 10/40; G16H 10/60; G16H 20/10; G16H 40/20; G16H 50/30; G16H 50/70

(57) ABSTRACT

A medical modeling system configured with twelve levels describing diagnostic, prognostic and therapeutic components. A plurality of methods within the modeling system involving genetic, proteomic, cellular and tissue aspects of pathologies. The modeling system identifies and describes molecular sources of pathologies. The modeling system also identifies therapeutic options. The modeling system makes predictions of disease progression with and without therapeutic intervention.

### FIELD OF THE INVENTION

The invention pertains to biological, biochemical, biomedical and medical modeling systems. Individualized medical modeling are computational duplicates of biomedical objects that delineate object structures, functions and interactions. Medical models apply computational chemistry and computational biology to represent, assess and test biological molecular and cellular dynamics. In addition to digital representation of microbiochemical models, medical models are also applied to organ, tissue, biosystem, body and population models. Individualized medical modeling is applied to medical diagnostics, prognostics, pharmacogenomics, in silico pharmacology and therapeutics. Medical modeling are an essential component of drug discovery, personalized medicine and precision medicine technologies.

### BACKGROUND OF THE INVENTION

The history of computer modeling began with NASA in the 1960s. In order to develop simulations of devices that could be computationally tested before remote activation in space, NASA developed a duplicate capsule to assess challenges. In 1970, duplicate capsule testing was activated when Apollo 13 encountered in-flight engineering challenges. NASA coined the term “digital twin” [DT] in 2010 to describe the computational model, typically a 3D model, of a physical object or device that could be digitally simulated and tested.

Today, DTs are mainly applied to electronic and industrial products. Computer aided design (CAD) and electronic design automation (EDA) companies apply software tools to design electronic devices with computer models. These tools are applied to designing semiconductors, electronic circuits and micro-electromechanical systems (MEMS). The devices and systems can be computationally modeled before production in order to work out problems before the real device is made.

Ansys (Synopsys) develops DTs for industrial devices and components in order to computationally test and simulate device mechanics. Nvidia also applies its GPU-based modeling to DTs for industrial object simulation. Cadence and Synopsys construct software products for application to electronic data automation applied to semiconductor design and operational testing. OpenEye Cadence Molecular Sciences, a division of Cadence, develops software for pharmaceutical companies to perform molecular biochemistry simulations; these simulations generally only represent static molecular structural models.

A variety of startups are developing DT software for application to various industries, from industrial and semiconductor design to healthcare systems. These include Twin Health (DTs for chronic disease management), Q Bio (radiological diagnostic DTs) and Unlearn.ai (DTs for drug trials). Q Bio is imaging constrained as it builds a passive model for simple disease detection; it's mobile device merely substitutes for a convention imaging device. None of these businesses are focused on the areas of modeling for personalized medicine diagnostics and therapeutics.

A group of new ventures (Genesis, Iambic and Isomorphic) are applying AI to find and accelerate small molecule drug discovery. This new generation of venture is seeking to discover existing biochemical compounds, not novel drug design.

IBM Watson for healthcare has developed AI applied to medical diagnostics. By analyzing large medical databases, the Watson algorithms identify patterns common in generalized pathologies. These symptom-based diagnostics were matched with off-the-shelf therapies, typically existing small molecule compounds. Watson does not focus on personalized medicine. This approach to computational biology may represent the apex of 20th century medicine.

In Virtual You (Princeton, 2023), Coveney and Highfield describe a history of digital twins with applications to biology, though they focus on general medical DTs performed with traditional supercomputers; they published their work before the advent of the GenAI revolution which makes possible the convergence of biological models with personalized medicine at the clinical level.

We are witnessing several converging technologies, including computer modeling for molecular, cellular, organ, tissue and systems simulations. These technologies typically apply to static physical structures.

AI, including generative AI, deep learning and machine learning, are applied to bioinformatics and to medical as well. AI is particularly useful for identifying patterns in big healthcare data sets, for learning about the behaviors of specific individuals and for predicting behaviors from past data analyses. Yet AI needs to be trained in order to make personalized medical predictions; so far, most of these predictions are focused on simple medical problems such as automating traditional medical diagnostics.

While the healthcare diagnostics field is becoming increasingly crowded, particularly with the advent of large language models (LLMs) and generative artificial intelligence (GAI) systems, these diagnostic approaches are focused on the macro level of general disease identification. For the most part, these systems are not focused on personalized medicine, which aims to develop a precise data analysis of each individual's genome and genetic dysfunctions and may require intensive computational resources.

### SUMMARY OF THE INVENTION

### Problems That the Invention Solves

Personalized medicine is the study of genomic variations applied to medicine. Assessment of a patient's unique combination of genetic mutations produces an insight in the precise source of a disease. Armed with this information, physicians are better able to accurately identify effective therapies.

Medical modeling is an efficient way to apply computational technologies, particularly modeling technologies, to solve challenges in personalized medicine. Medical models are classed into diagnostic challenges and therapeutics challenges.

In the case of diagnostic challenges, medical models provide accurate assessment of the molecular and cellular sources of patients'unique diseases. The invention describes different types of medical models for diagnostics to focus on each level of problem solving.

In the case of therapeutics challenges, medical modeling provides problem solving functions in order to analyze and select accurate treatments targeted to the specific molecular, genetic, proteomic, epigenetic or cellular sources of a patient's disease. The invention describes different types of medical models for medical therapeutics to focus on specific levels of therapeutic solutions.

One of the main challenges of realizing the vision of personalized medicine is that understanding the causes of disease at the genetic and proteomic level is extremely complex and computationally expensive. Only in the past couple of years have semiconductors and computers evolved to the point that computer modeling can be productive in order to emulate or assess complex biochemical molecular and cellular interactions and dynamics that result in disease. Yet, these computational modeling technologies have the potential to develop personalized diagnostics which lead to development of personalized drugs to target a unique set of patient genetic mutations that are tested in computer simulations before being actually applied to a patient.

Another challenge is identifying ways to computationally connect different scales of physical representations of molecules, cells, organs, tissue and biological systems.

These problems are solvable but require intensive computational technologies.

If the goal of medical science is to the provide optimum health wellness, the invention provides a blueprint for medical modeling that achieves the goal.

The invention develops a new approach to medical models for application to general medicine and to personalized medicine. The invention establishes a set of levels, or dimensions, of medical model focus. Several levels of models are developed for medical diagnostics and several levels are developed for therapeutics, while other levels of the system are developed for prognostics and unification of the levels.

For diagnostics, the different modeling levels focus on general wellness, molecular, cellular, organ and body level biomedical models. The medical models analyze bioinformatics data to identify precise structural and functional diagnoses of disease. There is also a prognosis medical model level, which predicts different disease evolution scenarios of a patient disease once a diagnosis is made.

For therapeutics, the medical modeling levels focus on problem solving in order to identify treatment options. In one mode, the therapeutics modeling levels interrogate the data in order to optimize and test the treatment options. In another embodiment, a therapeutics modeling level provides a dynamic prognostics analysis of different patient disease evolution scenarios based on application of therapeutic intervention options.

The system architecture described in the present invention is intended to be modular. Physicians and researchers can use one, several or all of the different levels of simulation technologies in order to optimize computational and economic efficiency and to optimize the patient's health.

Autonomous agents (intelligent software agents that are powered by chatbots and LLMs) are useful tools to assist physicians and researchers in collecting, organizing, describing and simulating medical data in medical models and simulations depicting healthy and diseased patients. The agents are particularly useful in assisting physicians and researchers to identify and simulate therapeutic options.

The medical modeling system is applied to general clinicians and to specialist physicians and surgeons, with numerous examples referenced. In addition, the medical modeling system is applied to medical research. Numerous recent experimental therapeutic modalities are described with reference to the medical modeling system as well.

### Advantages of the Invention

Individualized medical modeling for personalized medicine involves simulating biological molecular, cellular, organ, tissue and systems structures and functions. The advantages of medical model simulations include animating molecular and cellular interactions, identifying precise genetic mutations and their functional proteomics manifestations, developing customized biochemical or proteomic compounds or entities to treat unique genetic diseases, identifying and testing therapeutic options, identifying drug side effects and interactions, identifying disease prognosis and predicting therapeutic effectiveness.

It is possible, with enough diagnostic precision, for medical models to design a customized therapy for a particular patient condition. Additionally, medical models applied to biomolecular models facilitate the production of simulations to test therapy options in silico before implementing treatments in a patient. The medical models enable experimentation of therapeutics in order to interrogate the data and to perform a trial-and-error process to eliminate less useful options.

In addition to being useful for biochemical solution discovery, medical models are useful for tracking the therapy options in patients. Over time, the patient supplies updated tests to ascertain the efficacy of treatment options, which are then updated and optimized in the medical model in order to present supplemental treatment options.

While medical models for precision diagnostics is an invaluable component of the medical toolkit, the application of medical models to therapeutics is a crucial element of a personal medicine system. One cannot develop a precise treatment without having the benefit of precise insight of the diagnostic source of a disease, but the development of unique custom therapies to individualized biomedical challenges is the holy grail of personalized medicine. Medical modeling tools are uniquely suited to develop and test solutions to these complex biomedical problems.

Overall, medical modeling involves the interaction of AI and bioinformatics for application to personalized medicine in order to develop precise diagnostics and optimized therapeutics for disease management. The individualized medical modeling system endeavors to describe the mechanism of operation of each disease diagnosis in order to construct optimal therapeutic solutions.

The advent of modeling in medicine enables physicians to apply technologies that were once limited to corporate or government funded researchers operating with access to supercomputers. This development brings advanced computation to physicians and patients to allow the democratization of computer modeling applied to biomedical problems.

Reference to the remaining portions of the specification, including the drawings and claims, will realize other features and advantages of the present invention. Further features and advantages of the present invention, as well as the structure and operation of various embodiments of the present invention, are described in detail below with respect to accompanying drawings.

It is understood that the examples and embodiments described herein are for illustrative purposes only and that various modifications or changes in light thereof will be suggested to persons skilled in the art and are to be included within the spirit and purview of this application and scope of the appended claims. All publications, patents, and patent applications cited herein are hereby incorporated by reference for all purposes in their entirety.

### BRIEF DESCRIPTION OF THE DRAWINGS

For the purposes of illustrating the present invention, there is shown in the drawings a form which is presently preferred, it being understood however, that the invention is not limited to the precise form shown by the drawing in which:

FIG. 1 is a table of the multi-level medical modeling system;

FIG. 2. is a table of the medical modeling system showing relations between levels;

FIG. 3 is a flow chart showing a modeling level organized to identify unique genetic pathologies;

FIG. 4 is a flow chart showing the process of developing a model to diagnose a disease;

FIG. 5 is a flow chart illustrating medical models developed of multiple molecular structures;

FIG. 6 is a flow chart showing functional molecular interaction models;

FIG. 7 is a flow chart showing the process of developing a medical model of arteriosclerosis;

FIG. 8 is a flow chart showing the process of medical modeling to map the function of statins;

FIG. 9 is a flow chart showing the process of medical modeling to compare a patient's disease diagnosis to a medical database in order to predict the disease progress;

FIG. 10 is a flow chart showing the process of applying a medical model to identify therapeutic options to a pathology diagnosis;

FIG. 11 is a flow chart showing the process of applying an intelligent agent to identify and test therapy options;

FIG. 12 is a flow chart showing the process of applying medical modeling to generate and rank therapy options for a patient's unique disease;

FIG. 13 is a flow chart showing the medical modeling process of testing and ranking therapy options;

FIG. 14 is a flow chart showing the process of updating therapy options with feedback;

FIG. 15 is a flow chart showing the process of applying in silico experiments to model therapy scenarios;

FIG. 16 is a flow chart showing the medical modeling experimentation process involving in silico testing and in vivo testing;

FIG. 17 is a flow chart showing a medical model simulation of a drug's functional molecular pathways;

FIG. 18 is a flow chart showing a medical model prediction of therapy option outcomes;

FIG. 19 is a flow chart showing the process of updating a medical model and therapies with new information;

FIG. 20 is a flow chart showing the process of updating modeling of therapies and prognostic scenarios;

FIG. 21 is a flow chart showing the process of consolidating patient data in a medical model;

FIG. 22 is a flow chart showing the process of applying a database to a patient medical model;

FIG. 23 is a flow chart showing the process of applying a medical model to develop a patient diagnosis and therapy;

FIG. 24 is a flow chart showing the mechanics of applying algorithms to a medical model;

FIG. 25 is a flow chart showing the application of intelligent agents to medical models;

FIG. 26 is a flow chart showing multiple layers of diagnostic analysis in a medical model;

FIG. 27 is a flow chart showing the process of applying intelligent agents to build a medical model;

FIG. 28 is a flow chart showing application of generative adversarial networks to generate novel drugs;

FIG. 29 is a flow chart showing the process of applying two or more intelligent agents for diagnostic modeling;

FIG. 30 is a flow chart showing the application of intelligent agents to develop a medical model for therapeutic options;

FIG. 31 is a flow chart showing application of an intelligent agent to patient testing and therapy tracking;

FIG. 32 is a flow chart showing the process of applying an intelligent agent to collect patient data for a diagnostic assessment and therapeutic option assessment;

FIG. 33 is a flow chart showing the process of applying specialized medical modules to different medical specialties;

FIG. 34 is a flow chart showing the process of a surgeon applying a medical model to analyze a patient anomaly prior to a surgical procedure;

FIG. 35 is a flow chart showing the process of a cardiac surgeon applying a medical model to perform a medical procedure;

FIG. 36 is a flow chart showing the process of applying a medical model to analyze a pathogen and generate prospective therapies;

FIG. 37 is a flow chart showing a medical model applied to model ribosomal anomalies and therapies;

FIG. 38 is a flow chart showing the process of generating in silico experiments in a medical model to identify novel therapies;

FIG. 39 is a drawing of specialized medical modules derived from specialized medical databases;

FIG. 40 is a diagram of medical models and simulations of functional molecular interactions;

FIG. 41 is a diagram of software agents applied to diagnostics, prognostics and therapeutics in a medical modeling system; and

FIG. 42 is a diagram of a medical model of a molecular and cellular anomaly and the testing and reprogramming of gene and protein functions.

### LIST OF ACRONYMS

AI: Artificial intelligence
AGI: Artificial general intelligence
ASIC: Application specific integrated circuit
CAD: Computer aided design
CAR T cells: Chimeric antigen receptor T cells
CPLD: Complex programmable logic device
CPU: Central processing unit
CRISPR: Clustered regularly interspaced short palindromic repeats
CRISPR/Cas9: CRISPR-associated [protein 9]
CT: Computed tomography (scan)
DBMS: Database management system
DNA: Deoxyribonucleic acid
DRAM: Dynamic random-access memory
DT: Digital twins
EDA: Electronic design automation
FPGA: Field programmable gate array
GAN: Generative adversarial network
GenAI: Generative artificial intelligence
GPU: Graphics processing unit
IoT: Internet of things
iPSC: induced pluripotent stem cell
LLM: Large Language Model
MM: Medical Models
MEMS: Micro electro mechanical system
MRI: Magnetic resonance imaging
mRNA: Messenger RNA
mTOR: Mammalian target of rapamycin
OSKM: acronym for four (Yamanaka) transcription factors of OCT3, SOX2, KLF4 and MYC
PET: Positron emission tomography
PGx: Pharmacogenomics
RNA: Ribonucleic acid
siRNA: Small interfering RNA
SPECT: Single-photon emission computed tomography
SRAM: Static random-access memory
VR: Virtual reality

### DETAILED DESCRIPTION OF THE INVENTION

Personalized medicine is defined as a set of technological tools applied to medical diagnostics and therapeutics that identify and treat the unique genetic sources of disease. In the case of oncology, for example, each individual's cancer has a different combination of unique genetic mutations; when these genetic mutations are diagnosed, specific targeted therapies may be designed for these mutations.

In recent years, we have observed parallel revolutions in information technology and genomics. In the case of genomics, bioinformatics provides valuable insights into genetics, proteomics and epigenetics. Genomics plays a role in ninety percent of the top causes of death. In the case of information technology, data analytics and artificial intelligence provide insight into complex problems and provide access to accelerated solutions. These fields are converging. As an example, AI supplies tools for accurate medical diagnostics.

In addition to bioinformatics analytics, imaging diagnostics supplies valuable observable insight into diagnostic solutions. Here, too, AI supplies valuable analytical tools for pattern recognition of observable imaging diagnostics. Without imaging diagnostics tools, early disease detection would be hampered.

Medical imaging diagnostics include x-rays, ultrasound, magnetic resonance imaging (MRI), positronic emission tomography (PET), computed tomography (CT) and single-photon emission computed tomography (SPECT). Imaging diagnostics also includes endoscopic imaging techniques. Optimally, the digital data from these imaging technologies are input into a medical model for analysis.

In addition to imaging data, genetic data are input into the medical model in order to assess a patient's genetic profile. Biomarker data are also input into the medical model in order to assess a patient's proteomic anomalies.

The application of AI tools to personalized medicine is a natural one. While bioinformatics analytics supplies insight into dysfunctional genes, protein structures and dysfunctional proteins, applying AI to bioinformatics provides the acceleration of insights into observational genetic and proteomic patterns. Increasingly, biomarker tests are able to backward engineer the realization of individual gene mutations and their protein dysfunctions. AI applied to medical diagnostics is analogized to finding solutions to a complex puzzle with limited information.

Personalized medicine is increasingly focused on diseases that derive from genetic mutations, on orphan genetic diseases, on inherited diseases and on complex multi-variate diseases. Applying AI to solve these specific combinations of unique genetic challenges provides a clearer and more complete picture of the sources of diseases. AI also supplies tools for early disease detection.

The goal of diagnostics in personalized medicine is to obtain increased precision of the sources of diseases. In this sense, biomarker data supply genetic and proteomic dysfunction insights on the molecular and cellular levels. Only by understanding the source of a disease on a molecular and cellular level can precise therapies be identified. The unique details of each individual's specific health situation are identified by applying AI tools to precision medicine.

The paradigm case of the application of AI to precision medicine is the emperor of all maladies—cancer. Virtually all cancers are caused by some combination of genetic mutations. If we can identify these unique mutations, therapeutic solutions may solve the genetic dysfunction on the cellular level by applying correctly formed and precisely targeted proteins. Whereas it required millions of dollars to analyze an individual's genome twenty years ago as the field was emerging, it now costs less than a thousand dollars, with another similarly priced test for relevant biomarkers.

But much of medicine, beyond oncology, requires the discovery of genetic or epigenetic mutations as the source of diseases. Several thousand medical conditions have a molecular origin. About fifteen percent of genes, out of 30,000 active human genes, cause perceptible diseases from genetic mutations. For example, 2-6% of the population have a genetic disease. These diseases present a complex diagnostic challenge.

In many ways, the diagnostic medical challenges present only the initial set of problems for drug research. Once the genetic medical problems are identified, the solutions, in the form of drug discovery and medical treatments, become even more challenging.

What we need are tools from information technology applied to biological challenges. Bioinformatics supplies analytical tools for assessing large data sets, including genomic data. Even data on a single individual's genome can be massive. More recently, AI has been applied to bioinformatic analytics in order to identify complex patterns in massive data.

One emerging technology that enables deep analytics of genomic and medical data involves medical modeling. Medical models apply tools to duplicate each individual's medical data in a digital model. Increasingly, AI is applied to medical models in order to supply insight on the molecular, cellular and system level.

Individualized medical models take individual genetic, biomarker and imaging data in order to build a model of each individual medical condition. AI tools enhance medical model insights by building diagnostic models of specific diseases.

Medical models are well suited to assess disease prognostics as well. Medical models, guided by diagnostic inputs and by AI, are able to evaluate disease progression scenarios. Since diseases evolve in different directions and at different paces based on the behavioral or biochemical inputs, medical models are able to simulate the disease progression changes in models.

Medical modeling of disease prognostics is able to develop complex time-elapsed prognosis representations based on various inputs and assess vectors of disease evolution within a range of probabilities. For instance, a medical model on an individual disease can track biomarker assessment over time to evaluate probable disease progression.

Medical models are computer models. Medical models are a computational duplicate representation of objects that enable the visualization of biomedical structures from different angles and the testing of medical processes. In an active mode, as an example, medical models enable surgeons to map out and test surgical procedures before they operate on a patient.

AI models are computer programs that analyze data patterns by examining large data sets. An AI model is trained on data sets in order to analyze data patterns, detect anomalies, solve problems or make predictions from limited data. AI models use algorithms—symbolic code or mathematical language—to apply to a data set to make a decision. While an AI model can be used to make predictions or solve problems, algorithms apply the logic which the model uses to come to a conclusion. Consequently, AI models are used to automate learning and decision making, particularly when machine learning and deep learning techniques and algorithms are applied.

There are different classes of AI models, including generative models, discriminative models, classification models, regression models and foundation models. ML models are trained on data sets; by applying probabilistic analyses, the models learn. Foundation models (also referenced as base models) are pre-trained deep learning models trained on large data sets. These large language models, and the generative AI that rely on them, are a form of artificial neural networks and can have trillions of parameters. Large language models facilitate natural language processing (NLP), which analyzes and predicts LLM text patterns. These LLMs can be fine-tuned to specific AI applications. For instance, generative AI chatbots are derived from foundational LLMs; the chatbots are intelligent (autonomous) agents that apply generative adversarial networks (GANs). Intelligent agents are fine-tuned from generative AI (GenAI) to perform certain functions, such as problem solving, pattern matching or prediction. These AI operations are useful for application to medical models. As an example, GPT is an acronym for generative pre-trained transform. These GPTs, or transforms, enable the tracking of connections between proteins and genes. From the analyses of structural proteomic patterns, the transforms can make predictions of protein folding.

Machine learning is applied to medical models by applying algorithms, or instructions that provide a recipe for machines to analyze data, execute tasks and make decisions. AI algorithms are classified as supervised, unsupervised and reinforcement learning.

AI algorithms can be tailored for medical models to supply personal medicine solutions. Algorithms are applied to medical models for pattern matching, problem analysis, progression analysis, personalization and prediction. In the case of pattern matching, medical model algorithms are structured for problem finding, that is, to identify an anomaly in genetic or protein structures. The medical model algorithms are then configured for problem solving in order to analyze the problem, typically by applying classification and sorting techniques. The medical model algorithms personalize the medical model to a particular patient by fine-tuning the model in order to provide a level of customization; for example, the algorithms endeavor to match a cure to a specific patient disease. The medical model algorithms are organized to make predictions; in the context of prognoses of diagnoses and therapeutics, the algorithms analyze prediction scenarios within a range of probabilities. To make predictions, the algorithms apply progression analysis within the constraints of limited information. The medical model algorithms are programmed to automate processes with AI, ML, DL and GenAI techniques.

Guided by GenAI, medical models are even better able to identify risk scenarios—including the statistical chances of each scenario—very well. If a patient engages in unhealthy behaviors, a disease can be tracked in a negative scenario, whereas if a patient engages in healthy behaviors, a disease can be tracked in a positive scenario.

By applying inference algorithms, the medical models also supply risk-based predictions of patients and are able to supply healthy behavior recommendations. Disease progression scenarios can sometimes be contingent on specific inputs. medical models can then be applied to anticipate or predict specific disease scenarios based on different inputs. medical models can then update its prognostics scenarios with new data inputs.

While medical models and GenAI are useful for diagnostics and prognostics, they are also applicable to therapeutics. Medical models can also anticipate optimal health scenarios with application of precision therapeutics.

Before the discovery of DNA, it was impossible to trace the source of a disease to its cause. But with the deciphering of the human genome, we now have the tools to observe the genesis of disease with great specificity. Each of thousands of genes can be damaged in different ways and thus embody mutations that generate uniquely dysfunctional proteins. These dysfunctional proteins manifest in each individual's distinctive disease manifestation. Without understanding precisely which gene is mutated and exactly how this mutation is manifest, it is not possible to find a solution to this disease.

This is where medical models and GenAI are useful. Medical models require insight from large data sets including libraries of genes and proteins. Not only are medical models applied to the diagnostic challenge of finding the genetic problem of diseases, but the medical models are also useful in helping to identify the therapeutic solutions. GenAI is applied to medical models to recommend specific known therapeutic options; GenAI can also help a medical model to design a new therapy for a patient.

To date, there are several novel therapeutic modalities that are applicable to personalized medicine. These include gene editing (CRISPR), cellular programing (and reprogramming), stem cells, mRNA, synthetic biology, CAR-T, immunotherapies, mitochondrial therapy and protein replacement. Each of these therapeutic modalities are nascent, yet each requires a precise solution configured from an accurate diagnosis of a patient's disease.

A patient's precise disease diagnostic is just the beginning. medical models and GenAI are applicable to the diagnostics, but also to the precision therapeutics element. Once a diagnosis is precisely identified by a medical model, customized therapies are designed to solve the problem. A precision therapy is configured within the parameters of a personalized diagnosis.

Because precise genetic diagnostics with medical models present a complex anatomy of a disease, medical models are configured to recommend therapy solution options. Each scenario is supplied within a range of probabilities of potential outcomes. The challenge of medical models is to find an exact match of a precise medical therapeutic option to a very specific disease diagnosis.

Even the initial selection of a therapeutic solution option is just the beginning of attempts to solve the disease problem. medical models and AI are applied to tracking the therapeutics selection. New data on disease evolution, for example, from new or revised biomarker data, are provided to the medical model, which is constantly updated as the patient therapies are tracked. Drug side effects and drug interactions are tracked in the model as adjustments are made.

Whereas the traditional model of selecting an off-the-shelf medicine—typically a small molecule chemical compound—is a convenient starting point for medical model therapeutic selection to a precise disease diagnosis, the more long-term and personalized therapeutic solution lies in identifying specific personalized therapeutic solutions targeted to the exact disease parameters. Ultimately, the medical model will be able to design a personalized medicine for each precise disease diagnosis, like a custom key in a complex lock. Specific therapies are tailored to individual genetic variations. This is the holy grail of medicine, which is made possible only with medical modeling therapeutic solutions.

The challenge is to reverse engineer a specific molecular medicinal (genetic or proteomic) therapy in order to develop a personalized therapy to solve a precise genomic variation model problem. In some ways, this is a challenge of drug discovery with genetic precision on a micro scale. AI is well suited to apply to medical models to configure novel and unique biomedical or protein solutions to each specific unique genetic malady diagnosis. GenAI is particularly well suited for targeted or tailored drug discovery by matching unique gene mutation combinations to precise gene or proteomic therapies.

On a macro scale, the challenge is to find ways to tailor medications to treat patient's individualized genetic variations. For example, a targeted therapeutic regimen may include tailoring known drugs to specific combinations and/or types of genetic mutations. Medical models are useful not only for finding the best combinations of drugs for each individual's unique set of genetic anomalies, but they are also useful for tracking side effects, negative treatment outcomes and drug interactions.

Pharmacogenomics (PGx) is the study of how genome variations dictate a person's response to medications. Medical models and AI are applied to PGx in order to assess medicinal outcomes as applied to specific targeted diseases.

As an example of medical modeling, when viewed from a macro view, there may appear to be only a few different types of breast cancer. But when viewed from a micro view of precise genetic mutations, there may be hundreds of different mutations which may result in thousands of combinations of mutations that generate tumors. Each of these main categories of unique genetic mutation combinations requires precise targeted solutions. Viewed from this angle, breast cancer may be typified into hundreds of different categories, suggesting a very complex therapeutic challenge that requires many different precisely targeted genetic or proteomic solutions.

But therapeutics can be seen as a dynamic process. Therapies typically require iterative solutions, including tracking, updating, analyses and scenario development. Consequently, medical therapies can be seen as an experimental process, i.e., iterative, with feedback and necessary fine tuning. In a sense, this experimentation process with medical modeling—with trial and error and adaptation—typically continues until a therapeutic solution is found. Medical modeling reveals therapeutic probabilistic scenarios that enable adaptation of treatment options with the ultimate goal of curing the disease.

Biological, biochemical and medical experiments are conducted in silico by implementing medical modeling and analytics. The medical modeling test specific hypotheses in order to solve a problem. The medical models will employ software agents that apply algorithms for different analyses to specific medical problems.

In silico experiments with medical models are categorized as diagnostic, therapeutic and prognostic. For diagnostics, a medical model can perform virtual exploratory surgeries in order to ascertain information about a patient disease. The medical model can perform a digital biopsy or dissection procedure by analyzing diagnostic imaging data. The medical model can also analyze multivariate diseases by teasing apart the components of the disease, by applying protein structure prediction and by identifying differentiated molecular pathways of each respective disease. The medical model identifies each unique source of a patient's disease.

For therapeutics, medical models are applied to testing therapy options in silico. In effect, a physician performs an interventional therapy within the digital model. By first testing the different therapeutic options in the model, a physician receives feedback on the different therapy options. The feedback in the digital model may reveal unintended side effects or drug interactions that suggest a supplemental or alternative course of action. In an embodiment, a surgeon can perform a digital operation on a patient before actually performing the operation on the real patient. The interventional procedure can be custom tuned to the particular patient, much like a dress rehearsal before an actual show. This prepares the surgeon to focus on the patient's illness and may increase the efficiency of the actual surgical procedure.

In the case of prognostics, the medical model can perform experiments by predicting different outcomes based on specific medical variables of a patient's disease progression. The medical model assigns a probability to each variable of the diagnosis. For instance, if a patient continues an unhealthy diet, an ulcer or diabetes will evolve more rapidly. Diagnostic prognoses are not the only predictions that the medical models can perform. Physicians can project therapeutic intervention scenarios with medical models too. The model assigns probabilities to the outcomes of various interventionist options before the physician initiates a therapy in order to guide the choice of therapy. The model continues to develop outcome predictions based on continual follow up of the patient with new data. For example, after performance of an interventionist therapy, the feedback from the patient's recovery will inform the model to enable recommendations of updated therapy suggestions or probabilities of decline without therapeutic options.

Medical models are ideal for these sorts of therapeutic modeling of precision therapy scenarios that show progression with feedback and adaptation. The two goals of disease eradication and disease management are both fulfilled by medical models. While disease eradication is a clear goal, the most likely outcome of complex genetic diseases is disease management.

Medical models and AI are useful not only for prognostics of a disease once it is diagnosed, but also for therapeutic prognostics once specific therapies are initiated. Once a disease is tracked without a therapeutic intervention, the various scenario outcomes are probabilistically modeled in a medical model. But the disease is also tracked in a medical model after specific targeted therapeutics options are applied, with various options detailed with different outcome probabilities.

General solutions to healthcare challenges exist now. Many of the AI-based healthcare approaches are applied to develop simple 19^{th} century diagnostics of common diseases. Most of the AI-based approaches to therapeutics involve identifying and selecting off-the-shelf therapies to treat common diseases. In the main, these approaches involve applying AI to automate the existing, traditional, medicine paradigm.

For about twenty-five years, since the discovery of the human genome, there has been a new level of insight into the source of human disease. If ninety percent of disease has a genomic origin, with many diseases caused by a genetic anomaly, there is an opportunity to view medicine from a more precise viewpoint that considers the molecular sources of disease. Personalized medicine is the novel field that seeks to generate insight on the deeper causes of diseases from their genetic and proteomic origins. When a set of genes are damaged or mutated, they produce dysfunctional proteins. These malformed proteins operate in the machinery of cells and present as disease. Identification of the precise combination of genetic mutations reveals the exact source of each individual's disease manifestation. Consequently, this is the first step in identifying therapeutic solutions to the unique genetic malformations.

The personalized medicine revolution lies not only in identifying the correct mutated genes and the dysfunctional or mis-shaped proteins but also in finding with great particularity the unique therapy for each individual disease. Cancer is the paradigm for this model of personalized, or precision, medicine, but there are numerous other relevant fields that trace cardiac, neurological, immunological, hereditary and genetic diseases.

Yet, in order to maximize the potential of the personalized medicine revolution, it is necessary to develop tools that provide insight into precision diagnostics and therapeutics. So far, bioinformatics has applied genetic testing, biomarker testing and imaging data to identify genetic diseases. But none of these excellent technologies enable a complete picture of the problem. Without a clear diagnostic picture-on a molecular and cellular scale—it is not possible to identify therapeutic solution options to treat a disease.

Individualized medical models are likely to become the central—revolutionary—component of the medical industry as it evolves towards personalized medicine, yet are only beginning to develop in any useful way. Medical models enable patient medical information to be collected, analyzed and interrogated in order to identify precise diagnoses of diseases and to find, develop and computationally test accurate and effective therapies. Medical models are biological models that are enabled by AI technologies, including autonomous agents, generative AI, natural language processing, deep learning and machine learning.

As AI is rapidly developing, medical models are positioned to be the indispensable element in the physician's and medical researcher's arsenal. The convergence of AI with personalized medicine is embodied in medical models. Much as internet search or the smartphone became strategic technologies, medical models are the single most critical strategic technology in a rapidly developing medical industry that could consolidate around medical digital models as the most important and indispensable feature.

### Medical Modeling System Architecture

The present system of computer modeling is applied to medicine by categorizing different levels in the Solomon system architecture of individualized medical modeling. The system consists of twelve separate levels.

### Diagnostics: Levels 1-6

Level 1 [General Patient Model]
Level 2 [Bioinformatics Analysis]
Level 3 [Molecular and Cellular Description]
Level 4 [Structural Genetic Combination Pathology Identification]
Level 5 [Functional Molecular and Cellular Pathology Diagnosis]
Level 6 [Diagnostic Prognosis Simulation]

### Therapeutics: Levels 7-10

Level 7 [General Therapy Solutions]
Level 8 [Unique Therapy Solution Genesis]
Level 9 [Therapy Option Testing and Simulations]
Level 10 [Therapy Prediction Scenarios]
Level 11 [Unified Patient Model]
Level 12 [Human Population Model]
Level 1 [General Patient Model]

Level 1 combines general patient health data into a single package. This information may include a patient's genomic data, biomarker data, blood and urine test results, medical digital imaging data and medical IoT device data. For the most part, this initial level captures and collects data for a patient medical model. In addition, family history health data can be input into the patient base model at level 1. As patient health experiences develop, the model is updated.

The model contains the patient's health history in order to enable a physician to compare a patient's current health condition to earlier episodes. A physician can compare a patient's current illness to a previous healthy state by examining the patient history in the model.

This level is primarily anatomical, like a digital map. Typically, this level is managed by a physician. However, this general patient model can be accessed and controlled by the patient. As the patient moves between doctors, this general medical information presents an accurate picture of the patient. When a patient gets sick this general model represents a baseline.

In one embodiment of a level 1 MM, a patient chart supplies information to the patient's MM. Patient form data can be transferred to the MM. Autonomous agents can assist the patient to complete a form, the data from which is then transferred to the MM. Alternatively, a nurse or administrative assistant can work with the patient or physician in order to complete patient health information in the MM.

In some ways, this first level is for general MM modeling, which collects and presents patient data. We can view this first layer as observing a patient from the perspective of a wide-angle lens.

This first layer is also applied to maintaining general patient wellness. This level generates a general patient wellness status and provides a foundation for comparing later patient illnesses. For this reason, the medical MM benefits from a long-term relationship with a patient in order to track the patient over time.

When the patient maintains good health, the MM at Level 1 can be used as a point of reference for the prevention of illness.

### Level 2 [Bioinformatics Analysis]

This MM level provides a deeper bioinformatics analysis of the patient medical data and provides a report of specific patient maladies over time. A physician can interrogate this model to ascertain general information about a known patient condition. This level of MM provides a physiological evaluation of a patient and provides a summary of their medical records.

The bioinformatics analysis level is useful in consolidating diagnostic data in a patient's MM. The medical data are consolidated in order to build an anatomic model that includes a patient's genetic, biomarker and diagnostic imaging data. Each iteration of data over time is included in the model akin to slices of information that inform a complete evolutionary model. In addition to anatomical data, physiological data are input into the model by inclusion of various medical (e.g., blood and urine) tests.

The main idea of unifying data at level 2 is to enable a physician to have a snapshot of a patient on a deeper level than at level 1. While level 1 collects data into a general map, the map may exclude the level of detail in genetic or biomarker data. At level 2, however, the general model includes medical data to enable a physician to build different views of the patient. For instance, the physician can view different organ systems that are prone to illness while separating the healthy organ systems. By focusing on a patient's illness in specific organs or tissues, level 2 bioinformatics enables the physician to drill down on the source of a patient's disease. Once this analysis begins, the doctor can order more tests in order to fill in the model and obtain a clearer picture of the patient's condition.

### Level 3 [Molecular and Cellular Description]

Building on the previous levels, this MM level provides a detailed view of a patient's cellular and molecular data. This level provides insight into anatomy and physiology of a known patient pathology on a cellular and molecular level.

Healthy patients are used as a baseline for diagnosing illnesses. Medical data collected from a patient is evaluated by comparing the patient data to medical databases. Traditionally, the patient's disease is evaluated by identifying the patient's symptoms and running some medical tests. But with modern tools, we are able to identify the molecular and cellular sources of diseases.

For example, a simple diagnosis of a patient may reveal a diagnosis of arthritis. This patient would likely have pain in the joints. But there are over a hundred different types of arthritis. In most cases, arthritis presents as wearing down the tissue in and around joints. But there are different causes of arthritis that cause different manifestations and prognoses of the disease.

In order to identify the source of arthritis, it is necessary to understand the disease at the molecular and cellular level. Level 3 of the medical MM is suited for this analysis. A genetic analysis will reveal dysfunctional genes that create proteins that may affect the immune system. Antibodies attack the immune system itself in an autoimmune disorder. Level 3 models and tracks these molecular and cellular pathways of autoimmune disease that presents as an attrition of joints. But not all forms of arthritis have this genetic and proteomic pathway in cellular dynamics. Other forms of arthritis have a different mechanism of operation and presentation. Level 3 models are well suited for the analysis of molecular and cellular data that cause and present as varied mechanics in different diseases.

### Level 4 [Structural Genetic Combination Pathology Identification]

This MM level builds models in order to provide a diagnosis of the structure of a unique patient pathology. This level will include the three prior levels. Physicians can interrogate the model in order to identify a specific patient condition that was unknown before. The physician can run tests to verify the condition. A physician can request a second opinion from diagnostic analyses at this level.

Many diseases—from genetic diseases, inherited diseases and cancer—are attributable to identifying a unique combination of genetic mutations. Generally, these genetic mutations generate malformed proteins that operate in the machinery of cells and manifest as disease. For example, sickle cell anemia is an illness that generates from genetic mutations that configure malformed blood cells.

The MM level 4 is organized to identify these unique genetic combination pathologies by applying bioinformatics analysis of genetic and proteomic data. A patient's genetic and proteomic data are input into the model. The model then analyzes the genetic and proteomic data by comparing the patient data to a medical database in order to ascertain genetic anomalies. From these genetic anomalies, malformed proteins are identified.

Level 4 builds models of pathologies by applying AI and analyzing a patient's data sets and developing models to identify the structural attributes of a patient's disease. Once a patient's symptoms are evaluated, various diagnostic patient tests (blood, genetic, proteomic, biomarker, imaging, etc.) will assist a physician to identify a disease. The physician will develop a model to analyze the patient data in order to identify the sources of the disease. These disease origins may include genetic and proteomic data. At any rate, the MM will build a model of the patient's disease mechanisms that show the molecular and cellular pathways that describe the disease. From these clear models of the patient disease, the physician can generate an accurate diagnosis.

Level 4 develops models and simulations of multiple molecular structures, including non-coding genes, coding genes, RNA, DNA, chromosomes, telomeres and ribosomes. In some cases, the model will predict 3D protein structures from gene composition data. Not only will the modeling system develop models of healthy molecular structures, it will also model dysfunctional structures, including gene mutations, protein structural anomalies and protein structures that predict dysfunctional protein functions. The model will develop simulations of protein structural properties and make predictions of gene, RNA and DNA fragments and chains as well as 3D protein structure predictions.

The net result of the level 4 analysis is a precise diagnosis of a patient's disease. This level identifies the structure of a patient's disease on the molecular and cellular level.

### Level 5 [Functional Molecular and Cellular Pathology Diagnosis]

This MM level is designed to ascertain a patient diagnosis of a specific pathology on a molecular or cellular level. This level also identifies molecular and cellular pathways in order to trace the mechanism of a disease. Since many genetic or hereditary conditions are based on identifying a unique set of aberrant genes or dysfunctional proteins, this level is useful for clarifying these genetic sources of a patient disease. Level 5 modeling typically describes how a disease functions, from the genetic and proteomic source to the presentation of a disease manifestation.

One of the advantages of the genomic revolution is the ability to obtain individual medical information on the molecular level. These genetic and proteomic data sets can be analyzed by comparing data on larger populations. Genetic anomalies are identified by applying AI and model analytics in order to identify the sources of various patient diseases.

For example, a patient with breast cancer may have only 8 different genetic mutations as the source of their cancer, including all relatively mild mutations. A deeper analysis may reveal a familial genetic connection to breast or cervical cancer. On the other hand, another patient with breast cancer may have 73 different genetic mutations, including several extremely aggressive proteomic malformations, which indicate a much more aggressive form of breast cancer. These different analyses will inform the patients'MM and help doctors to configure different courses of treatments.

The modeling system at level 5 develops models and simulations of functional molecular interaction. These include (a) non-coding gene to coding gene interactions, (b) coding gene to protein interactions, (c) protein to protein interactions, (d) protein to ligand interactions and (e) protein to lipid interactions. The modeling system also develops simulations of ribosomal operations as ribosomes convert genes into proteins. The model may include intracellular behavior simulations as well as inter-cellular behavior simulations.

The medical model at this level can identify the genesis, contours, progression and scenario probabilities of the disease. In the case of breast cancer, the medical model can advise a physician about therapeutic options, including some combination of radiation, chemotherapy, immunotherapy and surgery (lumpectomy or mastectomy). With knowledge of the particularity of the genetic origins of the disease, the physician or surgeon can tailor a therapy to the patient.

An example of an application of level 5 is to Type II diabetes. Various symptoms will reveal the need for tests that result in a diagnosis. The diagnosis will feature a map of the molecular pathways and cellular mechanisms for the management of glucose and the need for insulin.

Another example involves arteriosclerosis. The MM builds a level 5 model from patient diagnostic data in order to assess the degree of buildup of arterial plaques. The level 5 analysis will examine patient HDL and LDL levels and develop a model of the pathways of molecular interaction of LDL levels. The model can analyze the structure of the patient's HDL and LDL on a molecular level. Because the buildup of plaques in arterial walls also affects blood pressure, these two interacting health conditions need to be tracked in the model. The level 5 model is able to present alternative drug remedy suggestions for each individual illness condition. Depending on the stage of the accumulation of plaque, the model can recommend various interventional procedures such as angioplasty, stent placement, coronary artery bypass grafting, peripheral artery bypass, carotid endarterectomy or vascular bypass. Alternatively, the model can recommend medicines, including statins, PCSK9 inhibitor, citrate lyase inhibitor, cholesterol absorption inhibitor or combination drug therapies.

Because arteriosclerosis is a progressive disease, it is necessary to track the patient over time. It is highly likely that the disease will never improve, which suggests that multiple additional therapeutic interventions will be required to manage the patient's condition. The physician will therefore likely return to the medical modeling system in order to develop further detailed analyses of the patient's condition.

As an example, statins function by reducing the quantity of cholesterol made in the liver and assisting the liver in removal of cholesterol in the blood. These mechanisms and pathways can be modeled in the medical model at level 5. Unfortunately, statins have significant side effects, including muscle pain, memory lapses, fatigue, low blood platelet count, kidney damage and dizziness. By tracking the operational mechanisms of molecular and cellular interactions manifest in high blood cholesterol, the medical model can develop an accurate diagnosis of the unique features of the patient's hyperlipidemia, detailing the probabilities of side effects of different statins for each patient relative to the patient's unique condition based on an analysis of their molecular and cellular interactions.

### Level 6 [Diagnostic Prognosis Simulation]

This level is designed to identify prognosis of specific disease pathways. The MM presents simulations in order to supply scenarios of possible disease progression based on different inputs. The MM is able to predict various scenarios of behaviors in its animations at this level.

Left alone, diseases progress at different rates under different conditions. For example, a patient that engages in smoking cigarettes may contract various lung disorders if they continue with the habit, while if they stop smoking, the chances of improving lung disorders may improve.

Level 6 is used to track a patient's history and to extrapolate scenarios of various disease outcomes from an analysis of this historical record.

The MM level 6 develops models to track patient illnesses at the molecular, cellular, organ, tissue, system and body dimensions. The model tracks molecular pathway vectors with different variables and inputs in order to identify and predict various future developments. If a patient's cancer is left untreated, it may continue to evolve to a next stage under specific behavioral conditions. On the other hand, if a particular intervention is applied to treat the cancer, the prognosis of the disease will be different than the base case of no intervention.

Level 6 compares the patient's disease state and genetic, proteomic and cellular data with a medical database. The medical model compares the patient's disease diagnosis to a medical database of many other patients with similar characteristics that encountered a similar disease. The medical model then projects forward the patient's likely experiences from analysis of the experiential outcomes of other patients in similar circumstances. The medical database comparison yields insights into the experiences of other patients with similar diseases and genetic dispositions. The model applies AI to identify different patterns in the analysis of comparing the patient's disease history to other patient's disease experiences. The model then makes predictions of patient disease evolution scenarios, with each scenario limited to a set of probabilities.

At level 6 prognostics, the disease progression is modeled without the application of medical treatments. This pure diagnostic prognosis analysis is aimed to present a baseline set of disease projection trajectories to physicians. This approach is a valuable tool in order to compare useful treatments to the nil option of no treatment on a disease.

Prognostics is a valuable tool for the patient and the physician in order to make predictions in the short run and in the long run. Once a diagnosis is made, particularly on a molecular and cellular basis in order to understand the source of a disease, providing realistic estimates of the direction of the disease evolution is helpful.

In a further embodiment, the level 6 prognostic simulation goes further than a simple diagnostic analysis and comparison with other diagnoses to assess probable predictions of disease progression. The present medical model also actively conducts in silico experiments in order to assess the most likely outcomes for different therapeutic options. Different kinds of cancer may yield various probabilistic outcomes if treated with chemotherapy, radiation, surgery or immunotherapy at different phases of the tumor development. This dynamic approach to medical modeling enables the model to match a targeted therapy to a precise diagnosis. With the prognosis analysis at level 6, therapeutic options can be modeled before they are implemented in the patient with a view to developing alternative disease treatment scenario options. Nevertheless, the prognosis evaluation at this level lacks the feedback of actual therapy implementation.

### Level 7 [General Therapy Solutions]

This medical modeling level applies methods to identify therapeutic solution options to a particular patient disease on a general level. Once a general diagnosis is made, this MM level enables physicians to select a therapy based on comparisons with similar common diseases. This level is also used to perform tests to ascertain a therapy's probabilities of success.

Level 7 is used to model general therapeutic solutions. Most small molecule medicines can be applied to the general therapy model. In many cases, a patient's symptoms lead to collection of general diagnostic data, such as bloodwork, which reveals a simple diagnosis. For instance, a simple bacterial infection may result in a treatment of an antibiotic.

At level 7, a software agent will work with a physician in order to collect and analyze patient data in a medical model. The medical model will first seek to make a diagnosis of the patient illness. The physician will identify the correct diagnosis and request therapy options from the medical model.

The medical model will apply AI and bioinformatics analysis tools to identify the patient's diagnosis in relation to many other patient's illness as referenced in a medical database. The database will reveal the outcomes of various therapeutic options that match the diagnosis under different conditions. The physician will prescribe a specific therapy that has the best chances of success in light of the patient's condition.

The model will seek to tailor the best available therapy to the patient's illness. For example, if the patient is on a variety of medications, it is important to prescribe a medicine that has some medical benefit but maintains the least chances of adverse drug reaction or interaction.

In an embodiment, once a patient's diagnosis is made, and discovering that the disease is a relatively paradigmatic illness that does not require a novel therapy, the modeling system at level 7 can design a drug composite from existing medicines. The modeling system can employ an AI empowered agent to search medical databases in order to find several different medicines that may be combined in order to solve a patient challenge. For example, a patient with a heart condition that includes both hypertension and hyperlipidemia may receive a recommendation for a unique set of cholesterol lowering and blood pressure medications, yet with the minimum side effects.

### Level 8 [Unique Therapy Solution Genesis]

This level enables physicians to design unique therapeutic solution options based on the results of a diagnosis of a patient's distinctive combination of genetic mutations. This advanced level can be useful in designing novel drug therapies. Pharmacogenomics (PGx) shows how genome variations dictate a person's response to medications. MMs, AI and PGx are applied at this level in order to assess medicinal outcomes as applied to diseases that feature a unique genetic profile. In addition, this level conceives of a treatment as a solution to a multivariate genetic optimization problem. In a sense, the therapeutic solution to a multivariate medical problem requires fine tuning an optimal treatment. A physician can seek a second opinion to therapy options at this level.

The medical model at level 8 is configured to enable physicians to develop a therapy to match a patient's unique genetic malady. Cancer is a paradigmatic application of this sort of analysis. But other therapies may also require tailoring a unique solution to a complex malady. For instance, autoimmune disorders, such as rheumatoid arthritis or lupus may require identifying the unique combination of genetic mutations in order to produce a specific targeted therapy.

There are various genetic disorders that also require specialized therapies that are tailored to the patient's unique genetic pathology. Once the medical model identifies the specific genetic mutations and proteomic dysfunctional manifestation at levels 4 and/or 5, the model develops a novel solution for targeting the source of the disease. Gene editing, protein replacement and RNA therapies are developed to treat the patient genetic illnesses, with medical models well suited to design optimal therapies.

Fewer than one third of human proteins are targeted by small molecules, mainly because of the similarity of protein structures that make it difficult to specify a protein target with particularity. Neither small molecules nor antibodies are effective in many cell membrane proteins. However, RNA therapies, including mRNA, siRNA and antisense oligonucleotides, may be good candidates for targeting some proteins or for inhibiting other proteins.

The medical modeling system is well suited to designing unique therapies. A GenAI powered intelligent agent identifies the genetic mutations and the proteomic anomalies of the patient's disease. The agent then compares the patient's genetic mutations and proteomic anomalies to at least one medical database. Once the agent accurately establishes a patient diagnosis, the agent develops a therapeutic model to solve the diagnostic problem in order to find ways to optimize the genetic mutations or the proteomic anomalies. The agent identifies gene editing as one therapeutic option and protein replacement as another therapeutic option. After assessing the therapy options via in silico experimentation, the agent determines the best course of treatment is implementation of a protein replacement protocol. The agent uses the medical model to design a novel protein structure and generate a novel drug discovery to precisely match the patient diagnostic medical challenge.

### Level 9 [Therapy Option Testing and Simulations]

This level allows physicians to provide animations of advanced therapeutic options. This approach enables physicians to experiment with different therapeutic options in order to test viable therapies. Pharmacogenomics (PGx)—showing how genome variations determine medication responses—and AI are combined to build medical models to assess medicinal outcomes as applied to specific targeted diseases.

For centuries, physicians relied on instinct and intuition in order to find patient cures and to manage patient diseases. On one level, medicine is about maintaining balance, i.e., optimizing a therapy for a unique patient malady or condition. In a sense, one goal of medicine is to assess all therapeutic options and to find a balance between options. We can conceive of healthcare as a sort of multi-objective optimization problem, with solutions seeking ways to manage a patient's condition over time. The medical modeling system is useful as an analytic tool to enable a physician to precisely diagnose a disease and then to develop therapeutic options. But these therapeutic options require some experimentation and fine tuning in order to identify the optimal solution for each patient. This insight reveals the artistic element in medicine. For example, diseases that involve the immune system may require a fine tuning that seeks a balanced optimization to promote excellent health.

In addition to developing a diagnosis and searching for therapeutic options, the medical modeling system is well suited to testing the various therapeutic solution options. Once the precise genetic or proteomic molecular analysis is performed in the model to identify the patient's unique genetic mutations and protein dysfunctions which form the root of the disease, the medical model produces a set of therapeutic option candidates at layer 8. At layer 9 these therapeutic option candidates are tested.

The medical model collects data from medical databases on many patients in order to compare the patient's precise diagnosis to other patients'conditions. There are likely to be many variables that distinguish the present patient diagnosis from other patients, though there are probably also some similarities. These similarities and differences of the patient's illness enable the construction of different levels of therapy for the disease. Various considerations are made in developing the therapeutic solution options, including potential side effects and drug interactions. A younger, relatively healthy, patient may have a different outcome to an interventionist therapy than a very old patient. Therefore, the therapy options are ranked according to the optimum outcome probabilities for each unique patient.

The MM tests the therapy options in the model by analyzing the prospective outcomes of each therapy option according to different environmental conditions. The model applies Monte Carlo simulation to prospective therapy candidates and sorts the therapy options according to the most probable likelihood of success for each therapy. The model then ranks the therapy options according to different preferences that are allocated by the physician in order to increase the probability of success in application to a particular patient.

The physician may apply the selected therapy to the patient. The patient receives the therapy and experiences the therapy over time, discovering feedback from the therapy option. The physician obtains more tests in order to track the patient illness as well as the therapy outcomes, which updated data are input into the model. From these updated data, the model then reevaluates the data on the evolution of the patient's disease and updates the therapy in light of the new data. The model may recommend an enhanced medicine, a new therapy, a combined therapy, a combined medicine and surgery or some other therapy solutions in light of the new evidence. In essence, the initial therapeutic modulation is only a phase of an experimental process.

With more patient data over a period of time, the medical model proceeds to develop a set of experiments in silico that include simulations or animations. While comparing the patient's therapeutic journey to a medical database of therapeutic options and outcomes, the model constantly updates the therapeutic scenarios for each patient. While one goal is to apply a therapy in order to completely cure a disease, increasingly, the goal of therapy is disease management.

An example of disease management may include thyroid disease management. Other examples include neurodegenerative disorders (e.g., Parkinson's disease or Huntington's disease) and neuromuscular degenerative disorders (e.g., muscular dystrophy).

The experimentation process employed by the medical modeling system involves both in silico testing and in vivo testing. The proposed therapy is compared to a database of other patient diseases. The various patient variables are examined and ranked. Each ranking of the patient variables is provided a probability. The medical model then selects and tests in silico the therapy options and re-ranks the options, repeating the process until a therapy is selected for the patient. Because the criteria of each patient vary from other patients, the therapy options are identified statistically.

In the case of testing therapy options on patients, a drug testing protocol is designed to track the relevant variables of a patient. The main concern is to track the efficacy of the medicinal, proteomic or biological therapy and to track possible side effects or complications. Particularly when a patient is taking multiple medicines, tracking for side effects is required. The feedback data from the patient is recorded and tracked by the medical model. As more data on therapy tracking continues, the therapy is modified as required until the disease is managed.

In an embodiment, the medical modeling simulation system can be applied to tracking drug functions on the molecular level. As a drug enters the blood stream, for example, the model can simulate the precise cellular absorption to show the functional molecular pathways of the drug's operation. A drug can be tagged and tracked in order to facilitate the tracking in an actual patient in vivo, like a chemical GPS tracking system. This tracking process can then be emulated in silico in the computer model simulations. These simulations are critical to understanding the biochemical processes and molecular interactions of various drug therapies. When applied to novel proteomic or gene therapies, this process of tracking therapies will accelerate our understanding of drug processes.

### Level 10 [Therapy Prediction Scenarios]

This level enables physicians to track different patient therapy solutions with feedback. With this level, physicians are able to update their therapy options. This level supplies MM simulations of predictive scenarios of various treatment options, enabling the modeling of prognoses relative to various therapeutic inputs.

This level tracks level 6, which provides prognostic scenarios to diagnostics. In the case of level 10, the modeling system has already obtained a diagnosis and has applied therapies. The therapy options are mapped and ranked. Scenarios are extrapolated in the model for each therapy option. The medical model accesses medical databases with massive data sets on patient therapies and patient outcomes. The model develops scenario predictions with various probabilities based on the most up-to-date patient illness data.

The model relies on analyses at level 8 for a targeted therapy genesis and an analysis at level 9 of the simulated experiments testing the various therapy options in order to develop a prognostic analysis of probable scenario outcomes. The patient's unique disease configuration is precisely identified and diagnosed along with various proposed therapy options. Yet, these early therapeutic options are a priori since they are not yet applied to the patient. The therapies are ranked in their likelihood of success and then applied to the patient according to the priority of a physician's preferences. The model predicts the outcome of the therapy options based mainly on the comparison of a patient to similar patients in a database.

Once they are applied to the patient, however, the therapeutic modality is activated. The patient receives feedback on the selected treatment, which draws more testing, which further updates the patient diagnosis. These evolutionary diagnoses track the applied therapies, which are then mapped and modeled.

These more dynamic interactive therapeutic applications with direct patient feedback are then modeled in order to develop a more accurate prediction of the outcome of the patient's disease. The probabilities of managing the patient's disease are modelled in the various scenarios as multiple differentiated therapies are applied and feedback to these therapies are received. This iterative process enables a physician to constantly update and fine tune the therapy in conjunction with recommendations of the medical model.

As an example of this process, some mental illnesses may be optimized with the medical modeling. In the case of bipolar disorder or schizophrenia, it may be necessary to constantly fine tune the patient's meds in order to optimize disease management.

### Level 11 [Unified Patient Model]

This level combines the previous levels into an integrated whole. Each of the multiple levels reveal specific dimensions of insight into an aspect of the human body. Level 11 views the body as a single comprehensive picture in which the individual layers can be aggregated in an analysis searching for an understanding of a complex multi-variate disease.

The medical modeling system can combine various levels in order to target a specific disease to diagnose and treat. Not every physician will require all of the levels for all medical illnesses. In fact, the system is designed for targeted application of only one or two layers at time in order to maximize computational and economic efficiency. However, it is ideal to combine all of the layers in the MM system into a single unified model for some patients. This systematic evaluation is particularly well suited for complex diseases or multifactorial diseases. In these cases, the medical modeling system is a crucial tool that will optimize personalized medicine.

The human body is complex. Over time, it will encounter disease challenges. As we age, we will likely need some therapeutic intervention. Particularly when we hit old age, we will likely need various therapies in order to manage end of life care. These complex diseases are increasingly linked to genetic differentiation that require personalized medicinal solutions for which the present MM system is optimally suited. For example, as we age, the immune system degrades, thereby making us susceptible to multiple infectious pathogens.

The present system in configured to be modular but also the be integrated with the aim to personalize medical care by providing computational and AI analytical tools to assess and provide diagnostics, prognostics and therapeutics on a molecular (gene and protein), cellular, organ, tissue, system and body level. When the different levels of the present MM system are combined, they enable the maximization of physician effectiveness by allowing the doctor to identify and solve hard medical problems and to customize medical solutions for each patient.

The combination of the various layers of the present MM system allows the doctor to model patient illnesses from the perspective of different dimensions. The physician can observe the general patient at level one like a wide-angle lens and then zoom into a very detailed analysis on the molecular or cellular level in order to make diagnostic discoveries on a personalized level. From this precision diagnosis, the physician then works to find solutions, from the generalized common twentieth-century solutions to the precision therapies enabled by advanced computational modeling technologies embodied in levels 8 and 9. Once the therapeutic options are selected or designed, the physician can then experiment with simulations in order to test the therapies in silico and, finally, select and apply the optimal therapeutic options. After receiving feedback from the patient's experiences with the selected therapies, the model updates its prognostics scenarios in order to provide insight to the model to update and optimize the therapy with a goal of curing or managing the disease. This iterative process is systemic and integrative, illustrating the unified approach to the MM modeling process.

### Level 12 [Human Population Model]

This level combines multiple individual medical models into a single sociological map. This level can isolate unique sets of individual patients in order to identify group diseases. While one patient can be immensely complex, particularly as we drill down on the patient's genetic and proteomic anomalies in order to discover the source of a disease and provide diagnoses, prognoses and therapies, the notion of applying the MM modeling system to a much larger population of patients is daunting. Nevertheless, there are applications for applying the MM modeling system to multiple patients.

For example, a family may share a genetic disease that can be traced by analyzing multiple family members in the medical models. Examples of these genetic diseases include Alzheimer's, cancer, heart disease, arthritis and sickle cell anemia, examples that typically involve one or more gene inheritance.

Epidemiological analyses can be performed on this MM level in order to trace causes and consequences of infectious diseases. Infectious disease vectors require modeling of multiple individuals over space and time. The medical modeling of infectious diseases is typically performed in a public health setting and includes a broad range of infectious diseases from influenza to sexually transmitted diseases. Most of these infectious outbreaks are controlled, but some may produce epidemics. Medical models are useful in modeling infectious diseases in a population and in predicting outcomes based on multivariate factors, such as distribution of vaccines or quarantine implementation.

Level 12 is also applied to public health modeling.

Overall, the different levels of medical models represent different “dimensions” of a single multi-dimensional computer model that captures different views or aspects of a patient's medical data. The unified patient MM represents a sort of digital map of the patient that elucidates and consolidates their medical history and present medical situation; such a MM will follow them for the rest of their lives. When a health episode occurs, the MM can drill down to the biological system, organ, cellular or molecular level in order to identify and solve a particular health disorder. These data are stored in the MM for future reference in order to help solve future health challenges. The consolidated medical models represent a library illuminating the history, present condition and possible futures of a patient.

The medical modeling system operates in a database management system. The DBMS can be open source or proprietary, but is generally maintained in the cloud as a software as a service (SaaS) data system. The patient data are stored in the DBMS for future reference. When the patient data sets are accessed, they can be analyzed and managed by a physician. The data storage is constantly updated by the physician as new data are added and new analyses performed.

In many cases, the medical modeling process accesses large medical databases to mine for and analyze information involving diagnostics, prognostics and therapeutics. In the case of diagnostics, the medical model will search for patterns to help to identify a disease. The model will then analyze and compare the disease in order to isolate and confirm the parameters of the disease. In many cases, there is a close match of a patient disease, such as heart disease or diabetes, with parameters of a disease discernible in the large data sets. In other cases, such as cancer, there will likely be an imperfect match of parameters of a search between a patient's genetic data and the database data. Similarly, in the case of passive prognostics, there is a matching of a patient diagnosis progression to the database data set.

In the case of a therapeutics search, the medical model will begin with the precise diagnostic analysis of a patient illness and then proceed to search for a therapeutic solution in large medical databases. In some cases, there is a good match between the individual illness and solution option recommendations in the medical database. In other cases, however, the precise genetic signature of the patient illness does not have a close match to medical database searches. In these cases, it is necessary to construct a novel therapeutic remedy. The medical model will construct a novel treatment option protocol by applying algorithms to analyze prior database therapy option examinations and evaluations. These novel therapeutic options are then tested in the patient. The active and dynamic prognostics of the patient disease evolution in the context of the therapy administration is tracked and updated similar to an experiment, with continued fine tuning of the treatment as more information is available on the patient's evolving condition and treatment modifications.

The mechanics of the medical modeling system can be described. The system applies data mining techniques to access large databases in order to identify hidden patterns. The system uses algorithms to seek patterns in data sets. For the most part, the system is applied to structured data in databases, but it may be applied to unstructured data (such as from IoT device data) with data object interpretation and translation processing. Various algorithms are applied to data mining, including Bayesian analysis, regression analysis, genetic algorithms, Markov chains, cluster analysis and anomaly detection (though this is not a complete list).

Machine learning (ML) is applied to search large data sets for patterns. Two main classes of objects that are searched in the database system for MM modeling are spatial and temporal objects. Spatial patterns include biological entities, such as molecules, cells, organs, organ systems and anatomical objects. These spatial patterns may also include genes and protein structures. Temporal patterns include functional biochemical trends in time series. These include molecular object functions such as protein functions or cellular operations. Prognostics—both diagnostic and therapeutic prognoses—analyze patterns of disease evolution. Temporal patterns are often viewed in the context of probabilistic scenarios. In either spatial or temporal contexts, ML is well suited for making predictions.

Interestingly, traditional ML models may outperform some GenAI models, particularly at prediction. Both ML and GenAI algorithms are applied to make predictions, within probabilities, from analyses of large data sets. For instance, ML and GenAI can predict protein structures.

GenAI, however, can also be applied to design protein structures. This graphic visualization process is useful in MM therapeutics analyses and novel therapy (drug) development.

GenAI is a novel technology for application to chatbots, which are typically a text-based form of intelligent software agent that use natural language processing (NLP) algorithms.

Intelligent agents are useful tools to assist physicians and researchers in the process of interacting with the medical modeling system. Agents are applied to diagnostics, prognostics and therapeutics. In the case of diagnostics, general practitioners and internists will apply agents to search for data in large medical databases. The agents will assist the physicians in order to build the patient model. The agents will apply various ML and GenAI algorithms to perform data mining in order to obtain the closest match of data patterns in a medical database to a patient disease data. In one implementation, agents can help a physician to interpret diagnostic tests for input into a medical model. For instance, agents can be useful in helping a physician to interpret digital imaging diagnostic data.

In a macro sense, the physician will begin the diagnostic process by seeking a general diagnosis from basic patient symptoms. But in some cases, the physician will use the patient's genetic data, particularly on genetic mutations, protein structure malformations and dysfunctional protein presentation to identify a precise diagnosis. The application of the agents, and their algorithms, to compare the patient data to the medical database data, will yield identification of a patient illness.

There are several layers of diagnostics. On the lowest layer of diagnostics, a physician may apply simple observational and analog test data (such as are generated from a stethoscope or blood pressure device). But the diagnosis is incomplete with this initial layer of information, so the physician orders more tests. In a middle layer of diagnostics, the physician may request blood tests and imaging data as simple tests for an initial, or provisional, diagnosis. But these limited test data are insufficient to make a precise diagnosis. At a higher level of diagnostic analysis, the physician will obtain genetic, imaging and biomarker test data to enable deeper insight into a patient disease. These advanced test data inform the medical models and requires intensive computational resources but also yields a more complete diagnosis. The medical model, in conjunction with intelligent agents, directs this process, operating as a useful medical aid to physicians.

In an example of the system, agents work with physicians and researchers to perform functions in order to develop medical models. The agents work with physicians and patients to collect patient medical information for input into the model. The agents work with physicians to efficiently develop accurate diagnoses to patient diseases. The agents work with specialists to develop therapy options and to apply therapy management protocols. The agents work with physicians and patients to make disease prognosis projections and predictions based on various therapeutic inputs. The agents work, finally, with specialists and researchers to develop novel therapeutic modalities that target precise diagnoses.

In one application, agents apply generative adversarial networks (GANs) to generate novel drug designs. For instance, a GAN powered agent may design a new protein structure to solve a malformed protein by comparing proteins to a structural protein database and generating novel protein architectural solutions. The MM will model and rank the protein solution options and select the best option. In general, intelligent agents optimize efficiency in implementing the computing and communications processes of healthcare management systems.

The medical modeling system is configurable to integrate multiple simultaneous intelligent agents. In part because the diagnostic, prognostic and therapeutic focused agents have different skills, they may be trained differently. For instance, the therapeutics agent may have several levels, including therapy identification (with optimization algorithm techniques) and drug design (with visualization techniques). In order to optimize the medical modeling system, the invention may be enabled by employing multiple agents at the same time. In these cases, two or more agents may work on a diagnostic problem in order to quickly find a precise patient diagnosis. Second, at least one diagnostic and at least one prognostics agent can work together to diagnose and simultaneously predict illness outcomes. Third, at least one diagnostic agent and at least one therapeutics agent can work together to identify a patient disease and then to identify therapeutic options. Fourth, at least one diagnostic agent, at least one therapeutics agent and at least one prognostics agent can work together in order to find a patient problem, treat it and then make outcome predictions. Fifth, at least two therapeutics agents can work together in order to find therapeutic solutions to complex diagnostic problems. Finally, at least one therapeutics agent can develop a novel therapy while a prognostics agent can work with the agent for in silico or in vivo testing in order to track the patient under different therapy options and provide probabilistic outcome scenarios.

In an embodiment of the invention, the medical modeling system develops a database, or accesses large databases, that organizes a typology of gene, protein and cell dysfunction. By narrowing typology options to different kinds of biochemical mutations, malformations or dysfunctions, the system optimizes its efficiency. Libraries of genetic and proteomic data reveal about 20,000 to 30,000 active genes with about 10,000 proteins that may have medicinal benefits. By comparing healthy gene, protein and cell data to anomalous gene, protein and cell data, we can evaluate a patient's disease data in the medical model. Access to these libraries of genetic and proteomic data will be the new gold of the era of precision medicine.

Once the general practitioner physician or internist has evaluated the patient and applied intelligent agents to assist in building a medical model by comparing the patient data to a medical database or library, a specialist physician will proceed to identify or develop a therapy. The specialist will engage an intelligent assistant (agent) in order to search medical databases, medical libraries, medical research literature and medical experiments in order to initiate the process of selection of therapy options. In many cases, the specialist will select an existing off-the-shelf medicinal therapy that will match the patient diagnosis. In some cases, the specialist will select one or a combination of available medications to treat a patient.

However, in many cases, particularly those in which the patient has a genetic disease with unique genetic mutation characteristics, it may be necessary to develop a novel therapeutic option. In these cases, intelligent agents may be useful to perform data mining in order to identify therapies that may be useful in developing a targeted therapy solution. The agents may also recommend, or design, novel or custom therapies that precisely target the patient illness. The agents may apply differential or non-linear equations to analyze the patient medical model in order to find solutions from unknown or incomplete data. The agents may apply techniques that reverse-engineer a protein architecture from an analysis of the proteomic or genetic source of a disease. The advantage of applying agents to work with physicians to solve complex problems is the agents apply algorithms that enable them to learn. This learning process is applied to design a novel therapeutic regimen to solve a complex patient illness.

Once the medical modeling system has assisted in developing a therapy, the physician can work with biotechnology or pharmaceutical companies in order to procure an existing chemical or biological therapy or to develop a novel chemical or biological therapy, or combination of therapies, within a class of FDA approved chemical or biological therapies.

The intelligent agents will work with the specialist in developing a medical model that includes multiple therapeutic options, each with a different probabilistic outcome. The physician then selects an optimal therapy option and applies the option to treat the patient. The treatment is then evaluated from feedback of the experiences and further testing of the patient. The treatment is iteratively updated in light of the patient's therapy feedback. An agent develops a prognosis analysis, within the constraints of statistical analysis, in light of the updated treatment data in order to guide the physician's therapeutic protocol.

Intelligent agents are also applied to work with patients. The agents interact between the physician(s) and the patients. For example, agents are useful in providing patient management tools. When a patient needs to manage medical testing, the physician may provide an agent to the patient to schedule and track the medical testing process. Similarly, in the therapeutic process, agents are useful to track patient post-therapy feedback and provide the information to the physician for detailed patient tracking. A physician can track the patient at a medical model dashboard in order to adjust the patient's medications, the details of which agents can facilitate.

GenAI, unlike traditional ML, has the advantage to enable graphics visualization. GenAI can present simulations or animations of healthy gene, protein and cell pathways. An animation is a functional representation of object relations over time. These animations can present different levels of simulations, from cellular interaction simulations and gene-to-protein simulations to protein-protein pathway simulations.

While it is useful to model and simulate healthy biochemical and cellular processes, it is optimal to model and simulate dysfunctional biochemical and cellular processes because this is the source of many diseases. These GenAI powered simulations are well suited to convert data from the medical model into precise diagnostic simulations to show the effects of specific genetic mutations on protein structure development and function as well as the effects of dysfunctional protein development on cellular operations.

In addition to these useful simulations, the medical modeling system is beneficial in applying GenAI to develop simulations of treatment options and prognostic probabilistic scenarios under different conditions. By generating simulations with GenAI powered agents, the medical modeling system investigates and conducts experiments to demonstrate proposed precision therapies.

AI is applied at each level of the MM spectrum. AI is applied, for instance, to the process of gathering healthcare data for a patient. AI is also applied to analytics in the context of problem finding in order to develop a precision diagnosis. AI is applied to the problem-solving context of seeking medical therapies. Finally, AI is applied to the prediction context of diagnostic and therapeutic prognoses in order to track a disease.

Not only is AI, including GenAI, neural networks, deep learning and machine learning applied to medical models, but different mathematical calculations are applied to medical models as well. These mathematical equations include algebraic calculations, differential equations and calculus. Calculus in particular is useful in order to identify temporal phenomena of molecular and cellular behaviors. Differential equations are useful for solving problems with incomplete information.

While the application of mathematics is critical to study medical models, the application of computers is critical as well. Specifically, particular semiconductors are useful to perform sophisticated modeling. These chips include GPUs, ASICs, FPGAs, CPLDs, CPUs, SoCs and neuromorphic circuits. In addition to these logic circuits, advanced memory circuits, particularly DRAM, SRAM and high bandwidth memory (HBM) circuits are useful computer hardware components. The advent of supercomputer level GPUs in particular in the last year have made possible the field of medical models. The Nvidia H100 (and H200) series [and GH100 (and GH200) [combine GPUs with CPUs]) series], the B100 series, the R100 series and X100 series (and beyond) are powerful circuits, arranged in ASIC arrays, with from eighty billion transistors (H100) and two hundred billion transistors (B100) to a projected trillion (or trillions) transistors in coming years. Also, GPUs from AMD (MI300 [150B transistors] and MI400 families, etc.) and Intel (Gaudi 3, Falcon Shores, etc.) are useful in developing modeling hardware. When combined with multiple advanced (version 3, 3E, 4, 4E, etc.) HBM memory circuits, these advanced logic circuits are beneficial in developing inference and training of large data sets.

A new era in computing began in 2023. Whereas before 2023, terabyte and petabyte scale computing were possible in order to generate medical models in discrete computing apparatuses, in 2023, the AI revolution changed the traditional computing paradigm. The advent of powerful GPUs enabled rapid analysis of data sets for GenAI modeling. After 2022, the computing paradigm focused on large data centers consisting of large GPU clusters capable of exabyte and zettabyte computing functionality. Millions of logic circuits, each possessing hundreds of billions and trillions of transistors, are now aggregated in vast networks of data centers. Instead of a single supercomputer calculating medical models for each individual's personalized medical model at great expense, we see data centers renting computer time to third party vendors that enable physicians and researchers to access computability in order to create sophisticated models and simulations. It is projected that within a decade about ten percent of electricity in industrial countries could be allocated to data centers. In light of these developments, the yottabyte (a million times exabytes, which are a million times terabytes) era is inevitable. In order to manage these massive data sets, it will be necessary to apply compression and decompression algorithms (as well as encryption and security algorithms) at computing and communications junctions in order to optimize real-time medical modeling.

While computer modeling, including medical modeling, goes beyond large language model (LLM) analyses, these LLM training and inference tasks represent a new paradigm in computer modeling functionality. Of course, medical models represent a graphic modeling approach, which is well suited to GPUs, FPGAs and ASICs. But the massive computing power only available in these recent advanced semiconductors enable the work that previously was performed by large supercomputers in weeks to be performed in a cluster of a dozen or fewer GPU chips in hours. As an analogy, while the human genome was decoded in 2000 at a cost of millions of dollars, currently a human genome can be decoded for less than $1000. The advent of a new generation of GPUs enables the possible construction of human medical models for less than $1000 in the next decade. Slices of a human MM (that is, small parts of a medical model accessed for a particular purpose) can be obtained and analyzed for less than $100.

Given the modular nature of the present medical modeling system, it is possible to model only one level of the system by physicians or researchers for a particular analysis in order to assist a patient for a reasonable cost.

### Applications of Medical Models to Physicians

There are a number of examples of physician clinicians or researchers that the present modular MM system is designed to assist.

A cardiologist can use the level 3 to assess an athletic thirty-eight your old patient for hypertension and then use level 7 in order to test the proposed medication since the wrong medication can trigger a dramatic drop in blood pressure during intense aerobic activity.

Another cardiologist can use level 4 to assess a seventy-year-old heart patient. The cardiologist can then use level 9 to track drug interactions of a proposed prescription with the patient's statins and blood pressure medication.

Still another cardiologist can use level 1 for a general assessment of a patient's cardiological and arterial condition as a baseline for future pathology examinations. The cardiologist can use level 11 when a cardiological pathology discovery is made in order to assess the pathology and identify and test the best therapy options.

A neurologist can use level 5 to assess a neurology patient by gathering test data and then test drugs in level 9 by initiating animations to show the trade-offs of specific meds with the patient's current meds in order to find an optimal drug and dosage for the patient's condition. The neurologist can also use level 10 in order to assess various drug therapies with feedback from the patient's experience by modeling the patient's drug interactions in order to identify probability-based prognoses scenarios of various proposed therapies.

Another neurologist can assess a patient's neurodegenerative disease to assess the causes of memory lapses. If Alzheimer's disease, typically in the form of amyloid plaque, is discovered at levels 2 or 3, the neurologist can then use Level 6 in order to map out prognosis scenarios based on hereditary, blood and genetic test data. Similarly, if Parkinson's or Huntington's disease is detected, the medical modeling system can identify the most probable causes and map out likely prognosis scenarios. Specific therapeutic options for neurodegenerative diseases can be developed and tested at levels 8 and 9 for each patient.

An epidemiologist can use level 12 in order to develop simulation models of possible epidemic dynamic configurations in a population under different conditions.

An internist can use level 1 for a basic patient MM. In this case, a physician assistant or autonomous agent can collect information from a patient's medical form, manual medical chart or electronic record. These data may include not only patient history but also patient hereditary information on relatives'health conditions. In addition, genetic, imaging and biomarker test data can be input. When a patient contracts an illness, these data are assessed as the physician applies other level 2 to level 6 diagnostic approaches in order to narrow down the precise cause of a disease. In most cases, once an internist discovers the precise nature of a patient disease and computationally models this illness, the internist then refers the patient to other specialist physicians or surgeons. In most cases, these specialists will then apply at least one of Levels 7 to 10 in order to computationally develop and test therapies in order to optimize the treatment. The specialist will prescribe a treatment and the system will track the patient's reactions to the treatment and the interaction of the treatment to other patient medications in order to fine tune the therapy.

Perhaps the paradigm of medical modeling optimal applicability is to oncology. After assessment of a precise diagnosis by medical models at levels 4 and 5, the internist will have an accurate description of the unique combination of genetic mutations that generate the patient's neoplasm. A referral to an oncologist will require identification and assessment of therapeutic options at Levels 8 and 9. In addition, after development of a unique therapy to treat the patient's cancer, the oncologist will use level 10 to track, assess and optimize the therapy. In many cases, the cancer is managed over the long run rather than fully eradicated. As the illness progresses, continued referral to levels 8 and 9 will yield refined and updated therapies.

Different types of medical specialists may tend to use different layers of the MM system. Since the system is modular, it is well suited to a la carte usage by different physician specialists and different types of medical researchers. For instance, specific physicians may tend to apply the prognostics layers on level 6 and level 10 in order to assess a patient's diagnostic and therapeutic prediction simulations. As another example, internists may only focus on a specific diagnostic layer. Other specialists may only focus on a specific therapeutics layer. Because the system is designed to be modular, it is maximally efficient to focus on one or two levels. On the other hand, for a complete analytical approach, level 11 combines Levels 1 through 10 in order to do a systematic evaluation, technical description of a disease and therapeutic development, simulations, testing, tracking and scenario predictions.

In an embodiment of the invention, the system enables different distinct modules for each medical specialty. These modules enable a specialist physician to take small bites from the system or from a single level of the system. For example, each module will access a different specialized medical database or library. A specialist in one field may not need the data about another field in order to perform their analyses. This approach provides a substantial focus of the system, enabling each specialist to efficiently zoom in on the data they need. Each module can focus on a specialization of only a few MM levels at a time in order to facilitate a specialist to solve a particular set of specialized patient challenges.

The main specialized modules focus on differentiated organ and system types. These include: heart, arteries and vascular system; brain and nervous system; oncology; lungs and pulmonary system; gastro-intestinal system; ear-nose-throat; ophthalmology; ob/gyn; endocrine system; immune system; orthopedics; etc.

The medical model system is applied to hospital management. Individual physicians, including internists, specialists and surgeons, integrate the medical modeling processes into their practices. The medical models are accessible from various computers and devices in order to enable ubiquitous access to patient information. The physicians access patient diagnostic data and large genetic and proteomic databases in order to efficiently perform diagnoses. The physicians also work to identify and develop therapies for patients by applying the medical modeling system. In many cases, the physicians will work with pharmaceutical, biotech and medical device companies in order to customize therapies for patients. Physicians in particular specialties will work with doctors in their respective specialty in hospitals in networks across the country and across the world in order to share ideas and advice on precision diagnostics and targeted therapies. But the medical modeling system is integrated into the practice of medicine. Medical modeling and precision medicine are therefore emerging as the central component of the medical profession, even more central than electricity.

Though the medical modeling system applies to human medicine, it can also apply to any eukaryote. Therefore, the system can be used by veterinarians and botanists.

### Applications of Medical Models to Surgery

Surgeons can use medical models in both preparation for surgery and in practicing surgery in real time. Medical models allow surgeons to study and analyze the nature of an abnormal anatomical or physiological feature. For instance, a surgeon can import diagnostic imaging data and biomarker data into a MM and analyze a patient anomaly. This preparatory analysis enables the surgeon to test a procedure or device in a MM before performing the procedure. While the surgeon is actually performing the procedure, the medical model can inform the surgeon in real time about probabilities of success of the procedure contingent on specific actions. Each procedure can be tested phase by phase, which is particularly useful for complex procedures that may benefit from a computational assistant. These applications of medical models to surgery may increase efficiencies, limit errors and reduce complications.

There are many and varied surgical methodologies that can be applied to patients in conjunction with medical models, including open surgery and minimally invasion surgery. Minimally invasive surgical techniques include endoscopy, arthroscopy, laparoscopy, bronchoscopy, thoracoscopy, colonoscopy, hysteroscopy, gastroscopy, etc.

Common cardiac surgical procedures to which medical models may be applied include heart valve repair or replacement, coronary artery bypass grafting, atrial fibrillation treatment, pacemaker installation, aneurysm repair and heart transplant. Interestingly, in cases in which a patient has had heart surgery but their disease has progressed, medical models are useful to track the disease progression and to assist physicians and surgeons on surgical and drug treatment plans and recoveries.

A cardiac surgeon can use levels 2 and 3 in order to develop a model of the patient's heart and vascular system before performing a procedure. The surgeon can perform tests on the model, including experiments, with different proposed actions. After the surgeon initiates a surgical procedure, diagnostic data can update the medical model in real time in order to supply the surgeon with more probabilistic prospects for specific techniques. The medical model can predict various outcomes based on the surgeon's procedure inputs. Particularly when the patient's disease requires a combination of surgical intervention and medicine, the medical model is useful to assist, at Levels 9 and 10, with providing drug therapies with various predictive scenarios.

An orthopedic surgeon can use level 9 and level 10 in order to simulate a prosthetic joint tailored specifically for a patient. The surgeon inputs the digital image data (x-ray or MRI) into the medical model in order to identify the precise optimal joint configuration. The system then simulates the precise prosthetic tailored to the patient and the data are output to a machine that configures the joint apparatus.

Medical models are useful for thoracic, brain, ENT, cancer and gastrointestinal surgeons as well.

Software agents may be particularly useful when surgeons are working with medical models. The agents function as a sort of automated medical assistant, featuring vast medical data access and analysis. Libraries of medical data are instantly accessible to the agents in a surgeon's particular area of specialty, enabling surgeons to provide a personalized approach to each patient disease. The agent's access to data informs the medical models. As the surgeon builds a medical model for each patient, personal data from diagnostic imaging and testing as well as aggregated data on many patient disease analyses help to inform the surgeon both before and after performance of surgical procedures.

For example, genetic testing of a patient may reveal a surplus of specific proteins or a deficiency of hormones or antibodies. These data will be helpful in order to inform the surgeon on the efficacy of performing a specific procedure to solve an illness and the probabilities of success of different procedural options.

### Applications of Medical Models to Biomedical Research

Merck's Keytruda is presently the world's top selling drug at $25B a year. Keytruda is a humanized antibody applied to immunotherapy intended to treat various (lung, skin, blood, stomach, cervical, breast, etc.) cancers. Keytruda (Pembrolizumab) is a monoclonal antibody which stimulates the immune system to destroy cancer cells. Medical models are well suited to diagnose cancer at Levels 4 and 5. From these analyses and simulations, the precise gene mutation sources are identified. Levels 8, 9 and 10 are then applied to ascertain the precise therapies for each patient by actively testing therapy options on the patient's immune system, including ways to target cancer cell receptors, as well as to predict prognostic scenarios in light of various therapeutic options. Next-gen therapies may be available by accessing a medical modeling system.

A biomedical researcher can use a composite of thousands of different patients'medical models in order to identify a baseline of median biological data of patients with a specific medical condition in order to test drugs in silico. The scientist can then use Level 9 and Level 10 in order to develop drug interaction scenarios in silico and in vivo.

Merck is marketing Winrevair, with a price per patient of over $200K a year, to treat pulmonary arterial hypertension (PAH). PAH, a form of hypertension that is generated from narrowing of arteries in the lungs, causes fatigue and shortness of breath and is more common in women of a certain age. The drug, which is administered as an injection, blocks the constriction of cells in the patient's lungs'artery walls. PAH can be modelled in a medical MM Levels 8, 9 and 10. Particles of the chemical can be simulated as interacting with patient pulmonary arterial constriction. In addition, simulations of the drug action can be made to optimize effectiveness either as a pill or an injection with nanoparticles. Winrevair has side effects, including formation of small skin blood vessels and hemorrhaging, which side effects can be simulated and anticipated. Optimal dosing of the drug can be simulated for each patient. Further, the drug can be simulated for drug interactions with other hypertension or cholesterol drugs.

Some drugs may work better when combined with other therapies. Other drugs may cause serious adverse reactions when combined. MM Level 10 can simulate these drug interaction potentialities.

Furthermore, new therapeutic modalities, such as mRNA-based cancer vaccines or immunotherapies, can be simulated in level 8 and level 9.

Gene editing is an example of an exciting new medical therapy to solve problems associated with genetic disease. This therapeutic modality has been applied successfully to sickle cell anemia, a painful disorder that deforms red blood cells. However, gene editing has a difficulty in real patient applications. These sorts of therapies can be simulated in levels 8, 9 and 10. In one example, CRISPR (and CRISPR-Cas9) can increase cancer risk and may have other unintended effects. By experimenting with the technique's application to specific diseases in computer simulations, researchers can save billions of dollars and years of time in order to discover novel cures with minimal side effects.

Small molecule drugs are the most applied drug therapies, because they can penetrate cell walls and can be easily targeted. However, small molecule drugs can also bind to unintended cellular targets, which often results in toxicity and adverse side effects. These drugs can be modeled in the MM simulations.

New therapeutic modalities involve biologics, which includes proteins, genes, RNA and antibodies. The biotechnology industry produces these biologics, which are biocomponents manufactured by living organisms. The application of these biologics has treated complex diseases. This class of drugs are ideal candidates for testing in MM simulations.

Similarly, medical models can be applied to cellular programming. Previously, most cell programming and reprogramming has been applied in vivo, which is expensive and time consuming. If we can engineer cells to activate genes or produce specific proteins on demand, this may lead to new therapeutic modalities. The challenge involves how to activate specific genes in cells without harming other cellular operations. Medical modeling and simulations provide an efficient method for testing the multiple gene-based and protein-based functions in testing new approaches to modifying cellular behaviors. One advantage of this therapy is that it can stimulate the body's own capacity to restore damaged cells. Cellular programming and reprogramming can be applied to neurodegenerative disease, musculoskeletal disease, cardiovascular disease, cancer and diabetes, collectively described as diseases of aging. Medical models can simulate the cell signaling processes within cells that are critical to understand as a preparation to reprogram cellular functions. New cellular programming therapies can be tested in MM simulations.

In an example of partial cellular programming, CAR (chimeric antigen receptor) T cells are modified T cells trained to recognize cancer cells. CAR-T cell therapy constructs CAR-T cells by isolating a patient's T cells and modifying them to express a chimeric antigen receptor. The modified T cells are programmed to target a receptor or antigen on a cancer cell, such as the CD19 antigen. By targeting a single antigen, the therapy cannot be effective if the antigen mutates. The first generation of CAR T cell therapy was like a sledge hammer, introducing side effects as its targeting mechanism would not only destroy cancer cells but other cells too. Additionally, this therapy may induce a cytokine swarm effect in which the patient's immune system would overwhelm the patient. A second generation of the therapy was intended to fine tune the programming of the T cells to direct them to particular receptors and to minimize side effects. For example, CAR-T therapy can be combined with drugs that minimize the effectiveness of the modified T cells if a cytokine storm is initiated. A third generation of the therapy is intended to combine CAR-T cells with genetic circuitry (such as gene editing) to activate or deactivate the T cells on command. The therapy can also direct the modified T cells to address other diseases, including immune system disorders and infectious pathogens.

While monoclonal antibodies are constructed by employing biological processes to attack targeted cells, CAR-T therapies reprogram a patient's own T cells in order to target particular cell receptors and to destroy malevolent cells or pathogens.

The medical modeling system enables physicians and researchers to model a patient's unique disease architecture and program the CAR-T therapy to tailor to the patient's disease.

The modeling system also enables the tracking and tuning of tyrosine kinase inhibitors (TKIs) to target specific genetic mutations.

If the cause of many diseases is the presentation of dysfunctional proteins, why not simply replace the bad proteins with correctly formed proteins? The protein replacement therapeutic modality may result in positive outcomes. Medical modeling is well suited to identify the proteomic dysfunction (and the genetic mutation genesis) as well as a novel proteomic solution. Synthetic biology provides tools for the generation of new customized proteins to replace existing dysfunctional proteins. This therapy is typically combined with lipid nanoparticles in order to deliver the replacement proteins to cellular nuclei. Interestingly, some RNA therapies may also be able to stimulate corrected protein, enzyme or antibody generation.

RNA therapies present opportunities to stimulate the body to activate genes to initiate various remedial functions. mRNA contains coded instructions to direct cells to generate a protein with its own natural cellular mechanisms. Once injected into cells, the cells read the mRNA like a recipe, generating specific proteins. In the case of infectious diseases, the mRNA can be tuned to force the cells to generate antigens that match a pathogen. The immune system identifies these antigens and generates antibodies to attack the antigens. By precisely matching the mRNA recipe to the pathogen, the mRNA molecules effectively teach cells to train the immune system to attack the targeted pathogen. Constructing the precise genetic code of the mRNA molecules is critical to programing the cells to generate antigens. HIV may be a candidate for mRNA therapy. In another application, mRNA may be applied (as a sort of vaccine) to target cancer cell receptors; cancer cells often trick the immune system, which, when made fully cognizant of the cancer cells, is enabled to perform its function of eliminating the cancer cells. The mRNA therapy is also applicable to immune diseases and to immunotherapies which train the immune system to attack invaders.

Medical modeling is a computational system suited to modeling mRNA therapies. After identifying the genetic structure of infectious pathogens, the modeling system can identify pathogen surface receptors that can be targeted for generating a blocking agent. The medical model generates a set of options of genetic code that can be configured to block the targeted receptors. The medical model then tests the various therapy options and ranks the options based on probabilities of success in achieving the investigator's goals. The medical model optimizes the genetic code after running a set of tests. The medical researcher then generates the mRNA therapy for application to patients. The mRNA is applied to patients, which then generate antigens that are matched to the pathogen and are recognized by cells in order to train the immune system. Theoretically, medical researchers only need a few hours after identifying a novel pathogen before they can test the mRNA code fragments for application to solve the infection.

Another therapeutic modality involves epigenetics, which activates or suppresses non-coding genes in order to regulate protein coding genes. If we can find ways to apply epigenetics to turn off dysfunctional genes that generate misconfigured proteins, then this may stop the development of diseases created from these malformed genes and proteins. Medical modeling and simulations are well suited to perform these analyses.

Another therapeutic modality involves mitochondria, the power plants within cells. As we age, these mitochondria begin to wear out. Declining mitochondria in white blood cells in particular generate a deterioration in our immune system's ability to defend itself. If researchers can find a way to maintain strong white blood cells (leukocytes, including lymphocytes, granulocytes and monocytes) by managing optimum mitochondria integrity, we may be able to program the immune system to control diseases including some cancers. Medical models are well suited to model these molecular and cellular behaviors. A new class of drugs may be developed to optimize white blood cell mitochondria health by simulating the various mitochondrial evolutionary scenarios as they interact with new therapies.

Still another therapeutic modality involves ribosomes. Ribosomes are protein structures within cells that operate as conversion factories with the main function to translate genes into proteins. When the ribosome conversion assembly is dysfunctional, it will convert genes into malformed proteins. Therefore, addressing the correct or optimal function of intracellular ribosomes may provide techniques for prevention of deformed proteins which are the source of diseases. Medical models will model the ribosomal architecture in order to assess its optimal functioning. The MM will compare the ribosome of a patient with a medical database in order to identify the ribosomal structural and functional anomalies. Once identified, the MM will endeavor to find therapies to correct the ribosomal anomalies. These may include gene editing, cellular reprogramming, protein replacement or mRNA therapies.

Another therapeutic modality involves anti-aging therapies. For the most part, the two main classes of anti-aging that affect medical models include rejuvenation (restoration of youthful phenotypes and features) and geroprotection (prevention of further damage). The field of aging is generally connected to the emerging field of epigenetics, which studies how behaviors and the environment affect the operation of our genes. A classic example of the application of epigenetics to aging involves exercise. Increased exercise, particularly aerobic exercise, appears to have positive effects on cardiac, pulmonary, neurological and muscular health. Epigenetic processes include methylation, acetylation, phosphorylation, ubiquitylation and sumolyation, the powers of which tend to decline with age. The decline of several physiological functions, including neurological degradation, cardiological degradation and muscular degradation, are symptoms of aging that require methods of rejuvenation. Still, while rejuvenation is a goal of anti-aging sciences, diet and exercise appear mainly to prevent, or slow, ongoing degradation.

The main categories of anti-aging sciences include telomere degradation, mitochondria degradation, immune system degradation, stem cells and synolytics. Medical modeling is well suited to analyze telomeres, including the modulation of telomerase (between the poles of regeneration and oncogenesis). As telomeres degrade, the underlying chromosomes change their integrity and cells die; therefore, medical models can analyze the process of extending telomere durability and thus improve cellular longevity.

Similarly, mitochondria-organelles found in most cells which facilitate biochemical processes of energy production-have their own DNA. When the mitochondria DNA degrade with aging, the power plant of cells declines and the cells are dysfunctional. In particular, the mitochondria in white blood cells diminish with aging, with the effect of compromising an individual's immune system. Once so compromised, the patient is susceptible to infection. Medical models are well suited to analyze these anatomical molecular and cellular processes.

There are several therapeutical strategies for cellular rejuvenation. These include stem cells, senolytics, mTOR inhibitors, gene editing and cellular reprogramming. Inductive pluripotent stem cell (iPSC) therapy represents a major advance in anti-aging science because via four transcription factors (OSKM factors [OCT4, SOX2, KLF4 and MYC]), cells can be rejuvenated to an embryonic state. Once retrained to an embryonic state, cells can be engineered into specific differentiated cell types. Interestingly, patients with iPSC enjoyed restored telomere length, restored mitochondrial function and positive phenotype observations. When iPSC therapy is combined with gene therapy, such as CRISPR-Cas9 gene editing, the findings suggest prevention and reversal of neurological diseases.

Senolytics studies senescent cells. Cellular senescence is described by the elimination of cell division. Senolytics are molecules that seek to induce death in senolytics cells and thereby prevent the appearance of aging.

Mammalian target of rapamycin (mTOR) regulates cellular metabolism and growth, while mTOR inhibitors block protein kinases. mTOR inhibitors, which are applied to the study of cancer, neurodegenerative diseases and autoimmune diseases, are also useful in anti-aging studies.

Cellular reprogramming involves a set of techniques, including stem cell reprogramming, and affects senolytics and epigenetics. Reprogramming cells to an embryonic state and then to a selective youthful cell type has been shown to affect cellular senescence and epigenetic methylation to induce repaired telomeres and mitochondrial function.

The medical modeling system is applied to these anti-aging studies by analyzing and optimizing molecular and cellular function and dynamics.

The differentiation of these MM levels in the medical modeling system enables providers to supply different services to physicians and to patients. In the lowest levels, patients may be able to possess their own general model that is then supplied on demand by physicians with the permission of the patient. This general MM map enables the encapsulation of a patient's biomedical map for constant reference. Healthy patients can maintain this lower level of MM as a comparative model.

On the other hand, the higher levels in the MM system can require rigorous data analysis and custom drug design that is computationally intensive. Providers may charge high fees for supercomputer time in order to facilitate these advanced diagnostic and therapeutic analyses.

The medical modeling system may apply to modeling therapeutic modalities as yet undiscovered or invented. In fact, the present invention is well suited to providing the tools for the invention of next generation therapies. When a diagnosis has identified an illness without a known therapy, the medical modeling system will experiment in silico until it has developed novel solutions for unique molecular or cellular problems. In an embodiment of the invention, the modeling system will combine multiple therapeutic modalities in novel ways to create a unique therapy for specific patients.

In an embodiment of the invention, a physician or researcher can access more than one level of the multi-layer MM system at the same time. This approach may be useful in order to analyze a diagnostic problem while also searching for a therapy. This approach may also be helpful to work with two or more patients at the same time.

When a physician works with more than one layer in the medical modeling system, they may be able to update data in all layers by updating data in one layer. The medical modeling system is integrated into database management system; a database input on one level can be duplicated in other parts of the DBMS.

For the most part, a physician can use level 1 as a general model layer that inputs health data from patient diagnostic tests to track a patient's general health. As the patient experiences symptoms of an illness, diagnostic tests may require a deeper analysis at Levels 2-6; but the data from the healthy patient at level 1 is imported into the other levels for comparative analysis.

In another embodiment of the invention, the medical modeling may be applied to medical devices and to the interaction of medical devices and patients. WHO defines a medical device as “any instrument, apparatus, implement, machine, appliance, implant, reagent for in vitro use, software, material or other similar or related article, intended by the manufacturer to be used, alone or in combination, for a medical purpose.” Medical devices are generally categorized as non-invasive, invasive or active. A medical device may include external devices, drug delivery devices and implantable devices, that are intended to diagnose, treat or inhibit a pathology.

Medical modeling can be applied directly to medical devices in order to describe, optimize and analyze the structure and function of the devices. However, medical models focus on the individual patient rather than the device itself. In this sense, medical models collect data and perform analyses of the patient interaction with medical devices. The medical models enable the testing of the medical device with regard to the personalized feedback of particular patients. The advent of medical models allows the development of personalized medicine by customizing and custom tuning the devices to individual patient's unique specifications. Consequently, medical models promote the development of customized medical devices that are fine tuned to a particular patient's unique needs and suited to solve the patient's unique disease. As the medical device registers inputs and outputs that affect the patient, the medical models can analyze the device processes to optimize patient outcomes.

In another embodiment of the invention, the invention applies virtual reality applications. VR is applied to the GUI in order to efficiently operate the medical models. A physician or researcher can wear a VR (or augmented reality) headset to access the medical model interface. The VR headset can control the medical model. Similarly, the invention is compatible with metaverse applications. By accessing a VR headset, the physician or researcher can access and control a medical model. This approach may provide valuable insights into molecular or cellular analyses.

Medical models transcend bioinformatics alone. Genomic data, biomarker data and bioinformatics analytics are useful ingredients for medical models. Taken alone, however, they are necessary but not sufficient for developing personalized medicine models of patient health disorders.

When a specialist uses the medical modeling system in order to accurately diagnose a disease and computationally identify a tailored medicinal therapy, the specialist may work with pharmaceutical or biotech companies to design the drug(s) specifically for a particular patient. In this sense, specialist physicians may work with pharma and biotech partners in order to perform tests of drugs in conjunction with the medical modeling system.

### DETAILED DESCRIPTION OF THE DRAWINGS

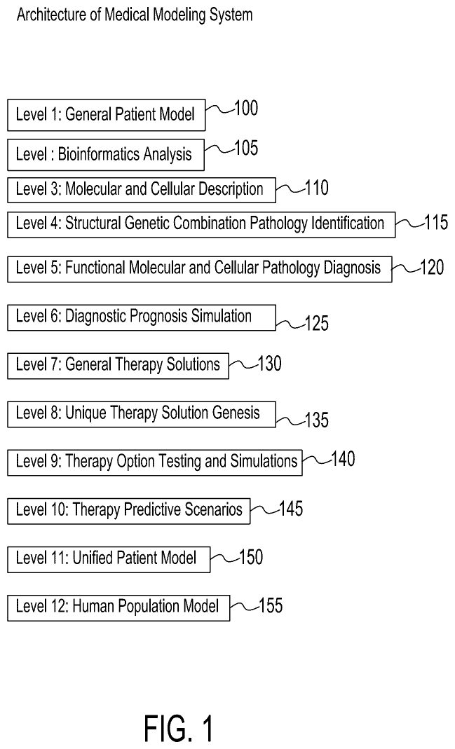

The present system of computer medical modeling consists of twelve levels. FIG. 1 is a table showing the architecture of the multi-level medical modeling system.
Level 1 (100) consists of a general patient model. This modeling level includes patient information that enables a simple disease diagnosis.
Level 2 (105) consists of a model that includes a patient's bioinformatics analysis. This modeling level includes bioinformatics data about a patient in order to assess a patient's disease diagnosis.
Level 3 (110) consists of a model that includes a patient's molecular and cellular description. Information about a patient's general molecular and cellular data are modeled at this level in order to assess a patient's disease diagnosis.
Level 4 (115) consists of a model that includes data about a patient's structural genetic combination pathology identification. The patient's genes are assessed, with data analysis about the combinations of structural genetic mutations that comprise a patient's pathology diagnosis.
Level 5 (120) consists of a model that includes data about functional molecular and cellular pathology diagnosis. At this level, information is modeled about functional molecular pathology, functional molecular networks and cellular networks that comprise a patient's pathology diagnosis.

The first five levels consist of diagnostic levels.
Level 6 (125) consists of models showing a patient's diagnostic prognosis simulation. A patient's untreated disease is predicted in diagnostic prognosis scenarios at this level.
Level 7 (130) consists of models of a patient's general therapy solutions. Available therapies are supplied at this level to provide treatments to diseases.
Level 8 (135) consists of models of a unique therapy solution genesis. A unique therapy solution is applied to a patient's disease at this level.
Level 9 (140) consists of therapy option testing and simulations. Therapies are tested and simulated at this level.

Levels 7-9 represent therapeutic levels.
Level 10 (145) consists of models of therapy prediction scenarios. When therapies are applied to treat a patient's disease, the therapy scenarios are predicted in models at this level.
Level 11 (150) consists of models of a unified patient model. In this level, the previous levels are consolidated in a single patient model or a model of models.
Level 12 (155) consists of models of a human population. Whereas the previous levels focus on models of an individual patient, models at this level focus on multiple patients in a public health setting.

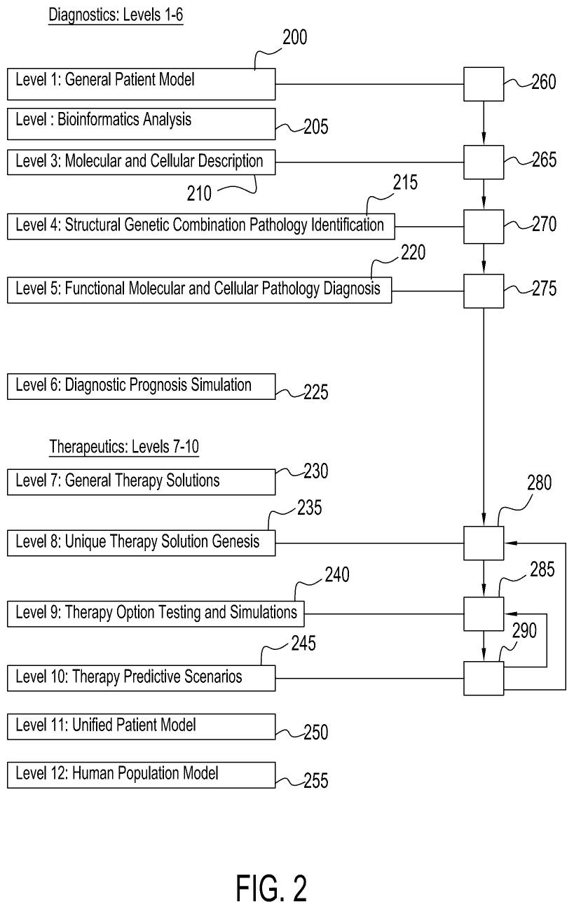

FIG. 2 is a table of the medical modeling system showing relations between levels. Levels 1-12 (200-255) are shown in the table. After collecting patient data to develop a model at level 1 (260), a physician may proceed to collect patient data and develop a model at level 3 (265), a model at level 4 (270) and a model at level 5 (275). These levels enable a physician to model a patient pathology in order to develop an accurate diagnosis. Once a precise diagnosis is developed, the physician will proceed to develop a unique therapy solution model at level 8 (280) and then test the therapy option(s) by producing model simulations at level 9 (285). The therapies are applied to a patient and therapy predictions are generated at level 10 (290) in order to assess scenarios of therapy utility with new patient data. The physician then returns to level 8 to generate additional therapy solutions options and to level 9 to test and simulate the updated therapy options. In one option, the physician moves from therapy prediction scenarios at level 10 to therapy option testing at level 9 and then returns to level 10 to predict an updated therapy.

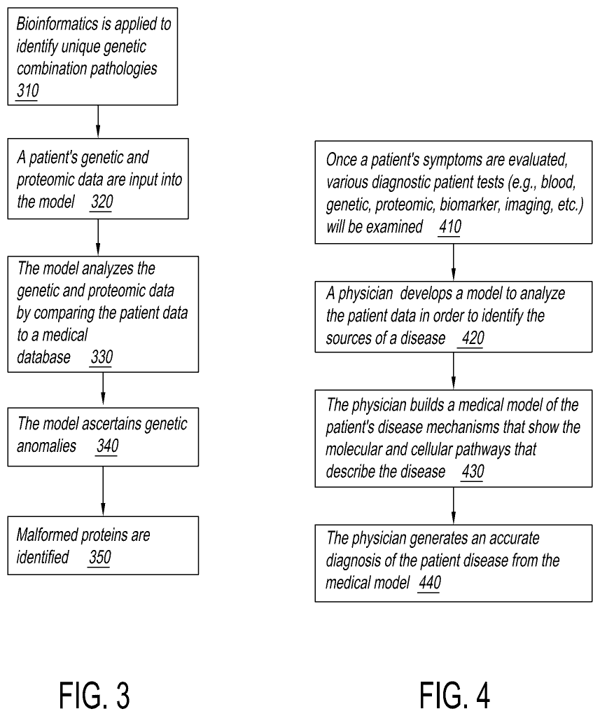

FIG. 3 is a flow chart showing a modeling level organized to identify unique genetic pathologies. Bioinformatics are applied to identify unique genetic combination pathologies (310) and a patient's genetic and proteomic data are input into the model (320). The model analyzes the genetic and proteomic data by comparing the patient data to a medical database (330). The model ascertains genetic anomalies (340) and malformed proteins are identified (350).

FIG. 4 is a flow chart showing the process of developing a model to diagnose a disease. Once a patient's symptoms are evaluated, various diagnostic patient tests (e.g., blood, genetic, proteomic, biomarker, imaging, etc.) will be examined (410) and a physician will develop a model to analyze the patient data in order to identify the sources of a disease (420). The physician will build a medical model of the patient's disease mechanisms that show the molecular and cellular pathways that describe the disease (430) and the physician will generate an accurate diagnosis of the patient disease from the medical model (440).

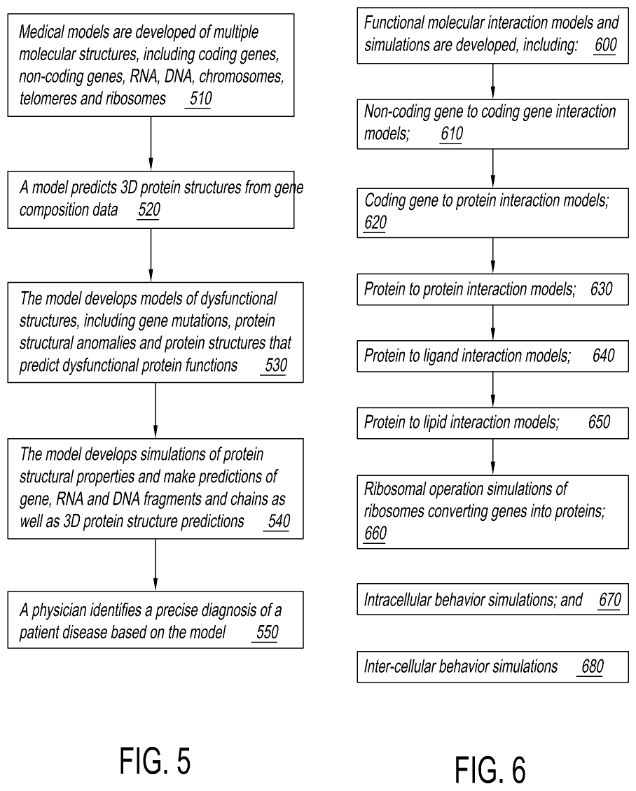

FIG. 5 is a flow chart illustrating medical models developed of multiple molecular structures. Medical models are developed of multiple molecular structures, including coding genes, non-coding genes, RNA, DNA, chromosomes, telomeres and ribosomes (510). A model will predict 3D protein structures from gene composition data (520) and the model will develop models of dysfunctional structures, including gene mutations, protein structural anomalies and protein structures that predict dysfunctional protein functions (530). The model will develop simulations of protein structural properties and make predictions of gene, RNA and DNA fragments and chains as well as 3D protein structure predictions (540). Finally, a physician will identify a precise diagnosis of a patient disease based on the model (550).

FIG. 6 is a flow chart showing functional molecular interaction models. Functional molecular interaction models and simulations are developed (600), including: Non-coding gene to coding gene interaction models (610), coding gene to protein interaction models (620), protein to protein interaction models (630), protein to ligand interaction models (650), ribosomal operation simulations of ribosomes converting genes into proteins (660), intracellular behavior simulations (670) and inter-cellular behavior simulations (680).

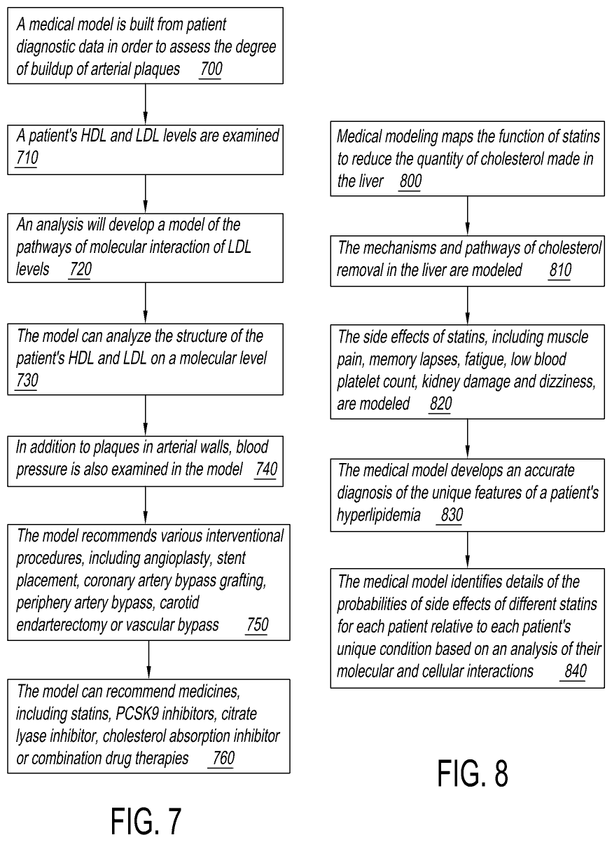

FIG. 7 is a flow chart showing the process of developing a medical model of arteriosclerosis. A medical model is built from patient diagnostic data in order to assess the degree of buildup of arterial plaques (700). After a patient's HDL and LDL levels are examined (710), an analysis will develop a model of the pathways of molecular interaction of LDL levels (720). The model can analyze the structure of the patient's HDL and LDL on a molecular level (730) as well as plaques in arterial walls and blood pressure (740). The model recommends various interventional procedures, including angioplasty, stent placement, coronary artery bypass grafting, periphery artery bypass, carotid endarterectomy or vascular bypass (750). The model can recommend medicines, including statins, PCSK9 inhibitors, citrate lyase inhibitor, cholesterol absorption inhibitor or combination drug therapies (760).

FIG. 8 is a flow chart showing the process of medical modeling to map the function of statins. Medical modeling maps the function of statins to reduce the quantity of cholesterol made in the liver (800). The mechanisms and pathways of cholesterol removal in the liver are modeled (810). The side effects of statins, including muscle pain, memory lapses, fatigue, low blood platelet count, kidney damage and dizziness, are modeled (820) and the medical model develops an accurate diagnosis of the unique features of a patient's hyperlipidemia (830). The medical model identifies details of the probabilities of side effects of different statins for each patient relative to each patient's unique condition based on an analysis of their molecular and cellular interactions (840).

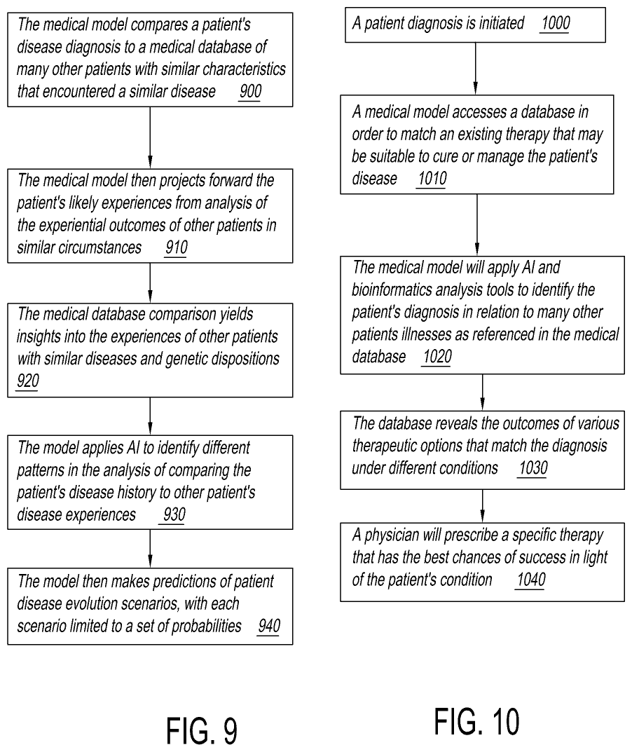

FIG. 9 is a flow chart showing the process of medical modeling to compare a patient's disease diagnosis to a medical database in order to predict the disease progress. The medical model compares a patient's disease diagnosis to a medical database of many other patients with similar characteristics that encountered a similar disease (900) and then the medical model projects forward the patient's likely experiences from analysis of the experiential outcomes of other patients in similar circumstances (910). The medical database comparison yields insights into the experiences of other patients with similar diseases and genetic dispositions (920). The model applies AI to identify different patterns in the analysis of comparing the patient's disease to other patient's disease experiences (930) and the model then makes predictions of patient disease evolution scenarios, with each scenario limited to a set of probabilities (940).

FIG. 10 is a flow chart showing the process of applying a medical model to identify therapeutic options to a pathology diagnosis. After a patient diagnosis is initiated (1000), a medical model accesses a database in order to match an existing therapy that may be suitable to cure or manage the patient's disease (1010). The medical model will apply AI and bioinformatics analysis tools to identify the patient's diagnosis in relation to many other patients'illnesses as referenced in the medical database (1020). The database reveals the outcomes of various therapeutic options that match the diagnosis under different conditions (1030) and a physician will then prescribe a specific therapy that has the best chances of success in light of the patient's condition (1040).

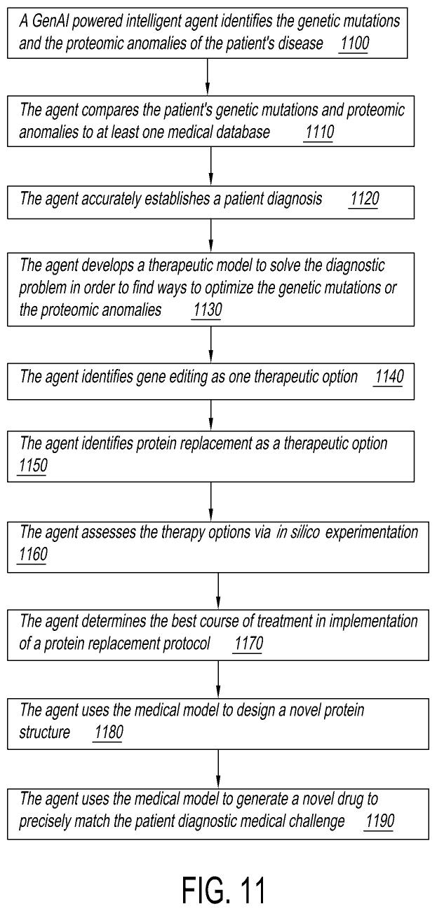

FIG. 11 is a flow chart showing the process of applying an intelligent agent to identify and test therapy options. A GenAI powered intelligent agent first identifies the genetic mutations and proteomic anomalies of the patient's disease (1100) and the agent compares the patient's genetic mutations and proteomic anomalies to at least one medical database (1110). The agent accurately establishes a patient diagnosis (1120) and develops a therapeutic model to solve the diagnostic problem in order to find ways to optimize the genetic mutations or the proteomic anomalies (1130). The agent identifies gene editing as one therapeutic option (1140) and protein replacement as a therapeutic option (1150). The agent assesses the therapy options via in silico experimentation (1160). The agent determines the best course of treatment in implementation of a protein replacement protocol (1170). The agent uses the medical model to design a novel protein structure (1180) and generates a novel drug to precisely match the patient diagnostic medical challenge (1190).

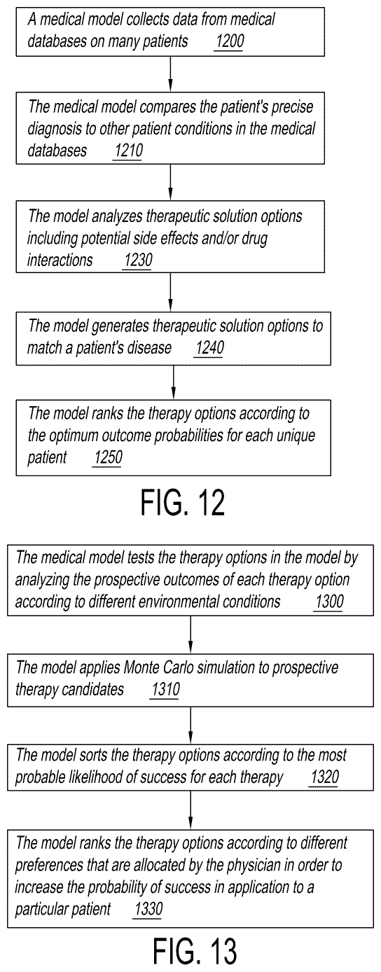

FIG. 12 is a flow chart showing the process of applying an intelligent agent to identify and test therapy options. A medical model collects data from medical databases on many patients (1200) and compares the patient's precise diagnosis to other patient conditions in the medical databases (1210). The model analyzes therapeutic solution options including potential side effects and/or drug interactions (1230). The model generates therapeutic solution options to match a patient's disease (1240) and ranks the therapy options according to the optimum outcome probabilities for each unique patient (1250).

FIG. 13 is a flow chart showing the medical modeling process of testing and ranking therapy options. The medical model tests the therapy options in the model by analyzing the prospective outcomes of each therapy option according to different environmental conditions (1300). The model applies Monte Carlo simulation to prospective therapy candidates (1310) and sorts the therapy options according to the most probable likelihood of success for each therapy (1320). The model ranks the therapy options according to different preferences that are allocated by the physician in order to increase the probability of success in application to a particular patient (1330).

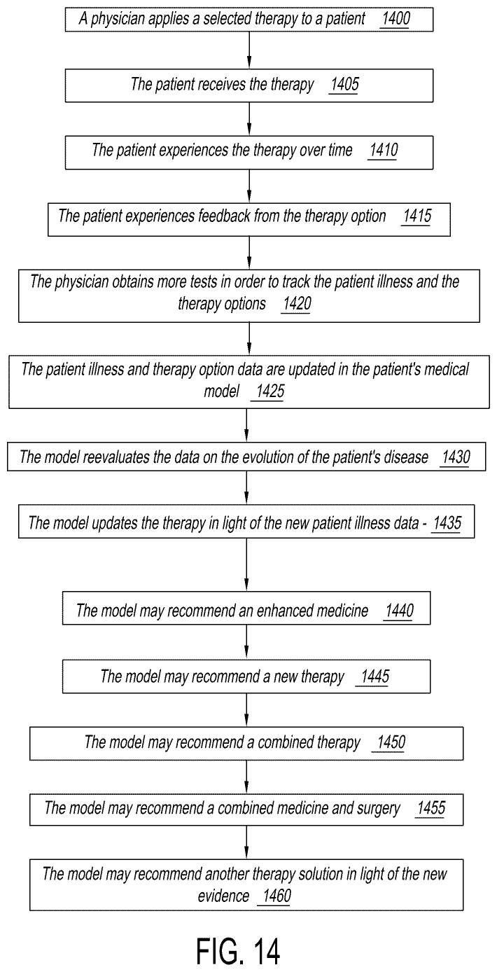

FIG. 14 is a flow chart showing the process of updating therapy options with feedback. A physician applies a selected therapy to a patient (1400) and the patient receives the therapy (1405). The patient experiences the therapy over time (1410) and experiences feedback from the therapy option (1415). The physician obtains more tests in order to track the patient illness and the therapy options (1420) and the patient illness and therapy option data are updated in the patient's medical model (1425). The model reevaluates the data on the evolution of the patient's disease (1430) and the model updates the therapy in light of the new patient illness data (1435). The model may recommend an enhanced medicine (1440), a new therapy (1445), a combined therapy (1450), a combined medicine and surgery (1455) or another therapy solution in light of the new evidence (1460).

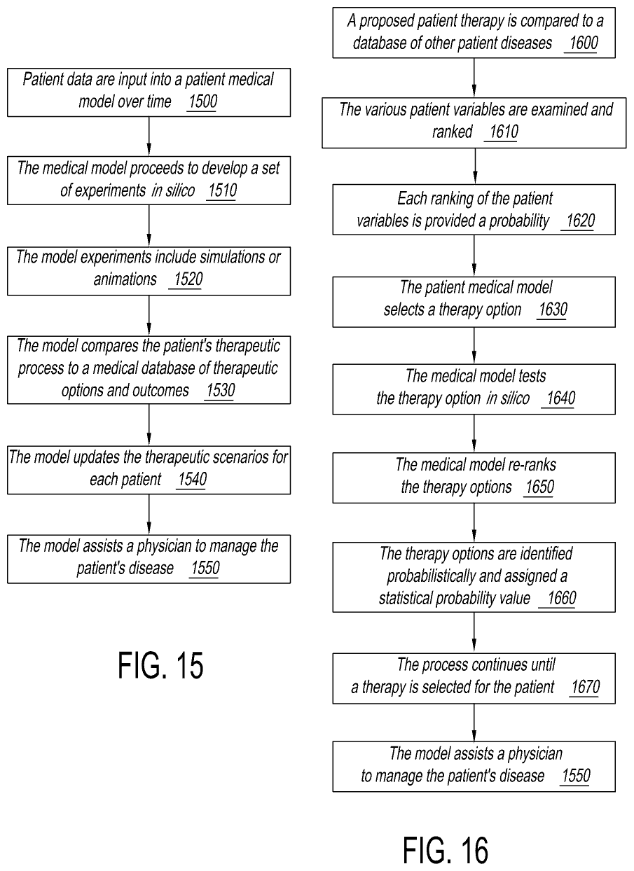

FIG. 15 is a flow chart showing the process of applying in silico experiments to model therapy scenarios. After patient data are input into a patient medical model over time (1500), the medical model proceeds to develop a set of experiments in silico (1510). The model experiments include simulations or animations (1520). The model compares the patient's therapeutic process to a medical database of therapeutic options and outcomes (1530). The model updates the therapeutic scenarios for each patient (1540) and assists a physician to manage the patient's disease (1550).

FIG. 16 is a flow chart showing the medical modeling experimentation process involving in silico testing and in vivo testing. A proposed patient therapy is compared to a database of other patient diseases (1600). The various patient variables are examined and ranked (1610), with each ranking of the patient variables provided a probability (1620). The patient medical model selects a therapy option (1630) and tests the therapy option in silico (1640). The medical model re-ranks the therapy options (1650) and the therapy options are identified probabilistically and assigned a statistical probability value (1660). The process continues until a therapy is selected for the patient (1670).

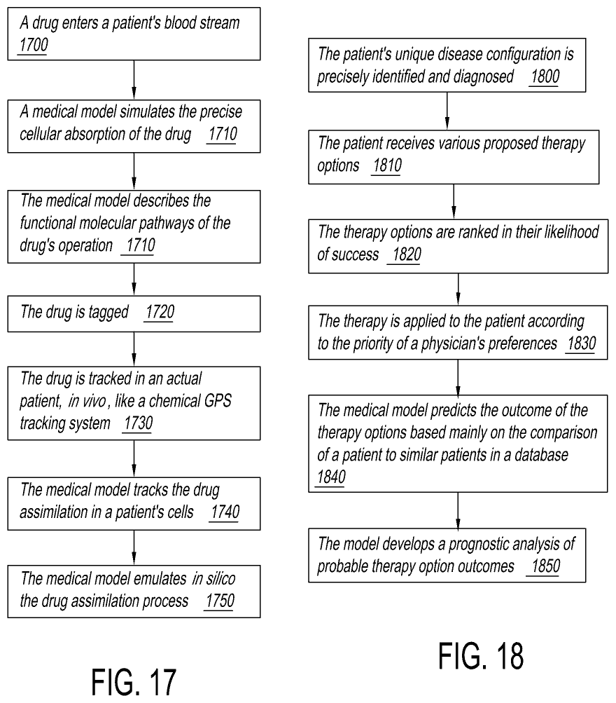

FIG. 17 is a flow chart showing a medical model simulation of a drug's functional molecular pathways. After a drug enters a patient's blood stream (1700), a medical model simulates the precise cellular absorption of the drug (1710). The medical model then describes the functional molecular pathways of the drug's operation (1710). The drug is tagged (1720) and tracked in an actual patient, in vivo, like a chemical GPS tracking system (1730). The medical model tracks the drug assimilation in a patient's cells (1740) and the model emulates in silico the drug assimilation process (1750).

FIG. 18 is a flow chart showing a medical model prediction of therapy option outcomes. The patient's unique disease configuration is precisely identified and diagnosed (1800) and the patient receives various proposed therapy options (1810). The therapy options are ranked in their likelihood of success (1820) and the therapy is applied to the patient according to the priority of a physician's preferences (1830). The medical model predicts the outcome of the therapy options based mainly on the comparison of a patient to similar patients in a database (1840) and the model develops a prognostic analysis of probable therapy options outcomes (1850).

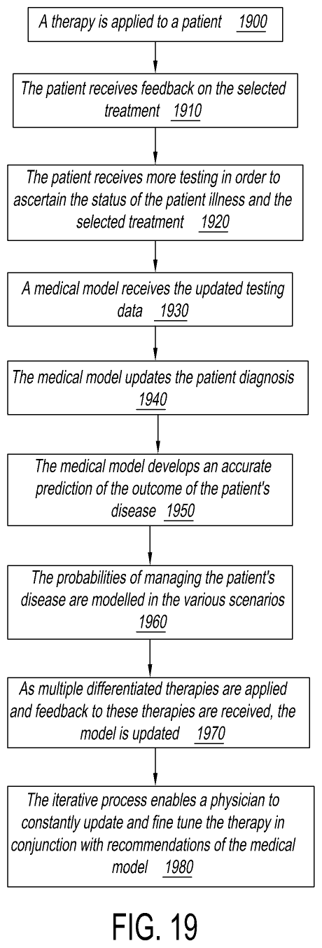

FIG. 19 is a flow chart showing the process of updating a medical model and therapies with new information. A therapy is first applied to a patient (1900) and the patient receives feedback on the selected treatment (1910). The patient receives more testing in order to ascertain the status of the patient illness and the selected treatment (1920). A medical model receives the updated testing data (1930) and updates the patient diagnosis (1940). The model develops an accurate prediction of the outcome of the patient's disease (1950). The probabilities of managing the patient's disease are modelled in the various scenarios (1960). As multiple differentiated therapies are applied and feedback to these therapies are received, the model is updated (1970). The iterative process enables a physician to constantly update and fine-tune the therapy in conjunction with recommendations of the medical model (1980).

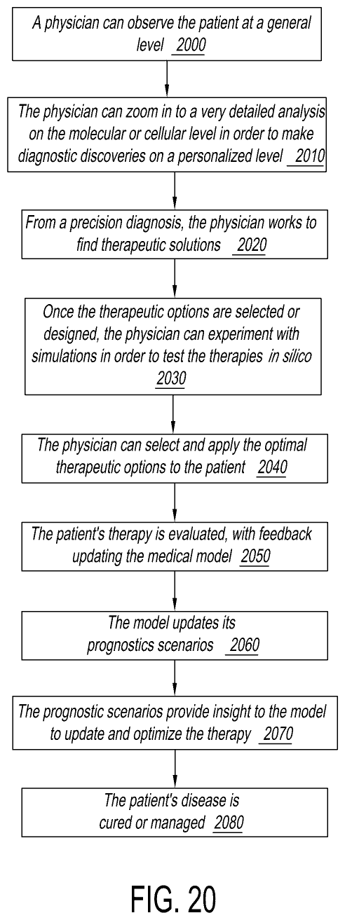

FIG. 20 is a flow chart is a flow chart showing the process of updating modeling of therapies and prognostic scenarios. A physician can observe the patient at a general level (2000) and the physician can zoom in to a very detailed analysis on the molecular or cellular level in order to make diagnostic discoveries on a personalized level (2010). From a precision diagnosis, the physician works to find therapeutic solutions (2020). Once the therapeutic options are selected or designed, the physician can experiment with simulations in order to test the therapies in silico (2030). The physician can select and apply the optimal therapeutic options to the patient (2040) and the therapy is evaluated, with feedback updating the medical model (2050). The model updates its prognostics scenarios (2060) and the prognostic scenarios provide insight to the model to update and optimize the therapy (2070). The patient's disease is cured or managed (2080).

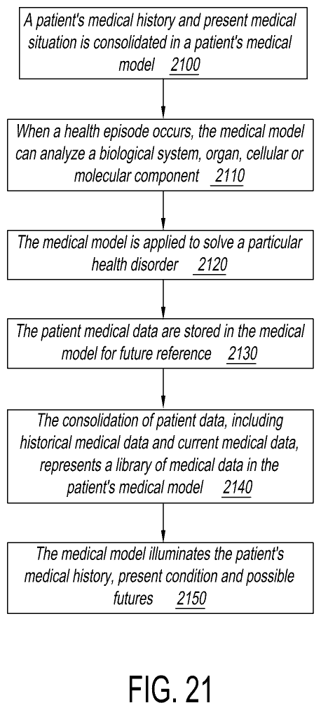

FIG. 21 is a flow chart showing the process of consolidating patient data in a medical model. A patient's medical history and present medical situation are consolidated in a patient's medical model (2100). When a health episode occurs, the medical model can analyze a biological system, organ, cellular or molecular component (2110). The medical model is applied to solve a particular health disorder (2120) and the patient medical data are stored in the medical model for future reference (2130). The consolidation of patient data, including historical medical data and current medical data, represents a library of medical data in the patient's medical model (2140). The medical model illuminates the patient's medical history, present condition and possible futures (2150).

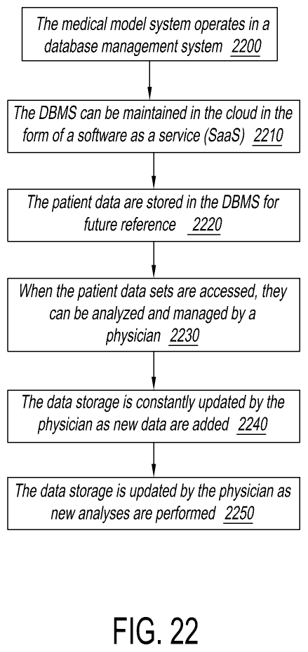

FIG. 22 is a flow chart showing the process of applying a database to a patient medical model. The medical model system operates in a database management system (2200), while the DBMS can be maintained in the cloud in the form of a software as a service (SaaS) (2210) and the patient data are stored in the DBMS for future reference (2220). When the patient data sets are accessed, they can be analyzed and managed by a physician (2230). The data storage is constantly updated by the physician as new data are added (2240) and the data storage is updated by the physician as new analyses are performed (2250).

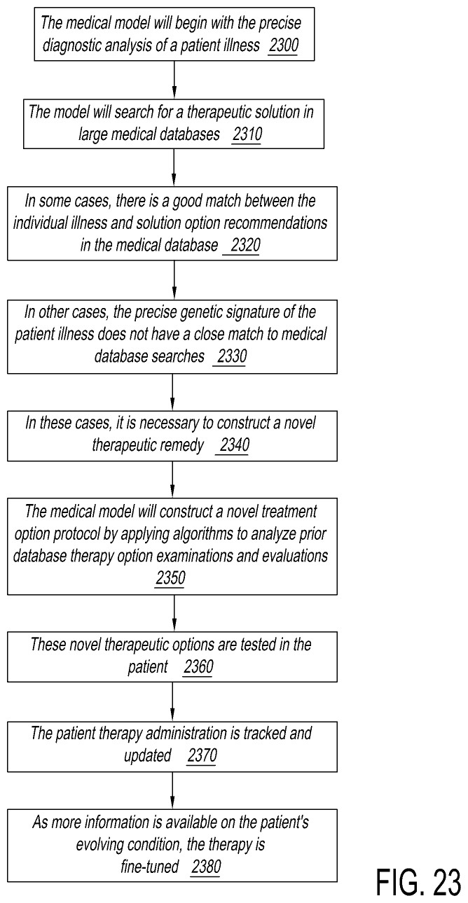

FIG. 23 is a flow chart showing the process of applying a medical model to develop a patient diagnosis and therapy. The medical model will begin with the precise diagnostic analysis of a patient illness (2300) and will search for a therapeutic solution in large medical databases (2310). In some cases, there is a good match between the individual illness and solution option recommendations in the medical database (2320). On other cases, the precise genetic signature of the patient illness does not have a close match to medical database searches (2330) and in these cases, it is necessary to construct a novel therapeutic remedy (2340). The medical model will construct a novel treatment option protocol by applying algorithms to analyze prior database therapy option examinations and evaluations (2350). These novel therapeutic options are then tested in the patient (2360) and the patient therapy administration is tracked and updated (2370). As more information is available on the patient's evolving condition, the therapy is fine-tuned (2380).

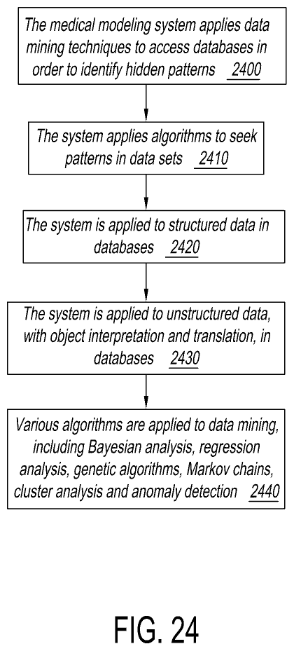

FIG. 24 is a flow chart showing the mechanics of applying algorithms to a medical model. The medical modeling system applies data mining techniques to access databases in order to identify hidden patterns (2400). The system applies algorithms to seek patterns in data sets (2410). The system is applied to structure data in databases (2420) and is applied to unstructured data, with object interpretation and translation, in databases (2430). Various algorithms are applied to data mining, including Bayesian analysis, regression analysis, genetic algorithms, Markov chains, cluster analysis and anomaly detection (2440).

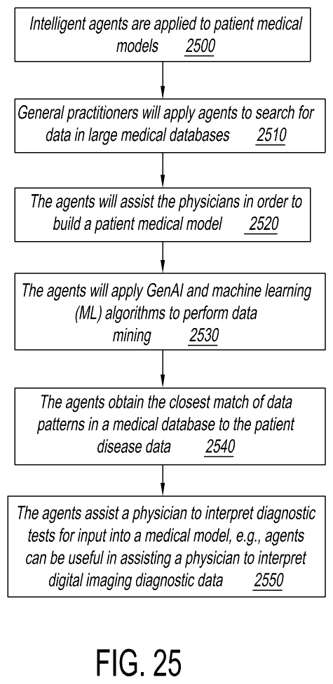

FIG. 25 is a flow chart showing the application of intelligent agents to medical models. Intelligent agents are applied to patient medical models (2500). General practitioners apply agents to search for data in large medical databases (2510). The agents will assist the physicians in order to build a patient medical model (2520) and will apply GenAI and machine learning (ML) algorithms to perform data mining (2530). The agents obtain the closest match of data patterns in a medical database to the patient disease data (2540). The agents assist a physician to interpret diagnostic tests for input into a medical model, e.g., agents can be useful in assisting a physician to interpret digital imaging diagnostic data (2550).

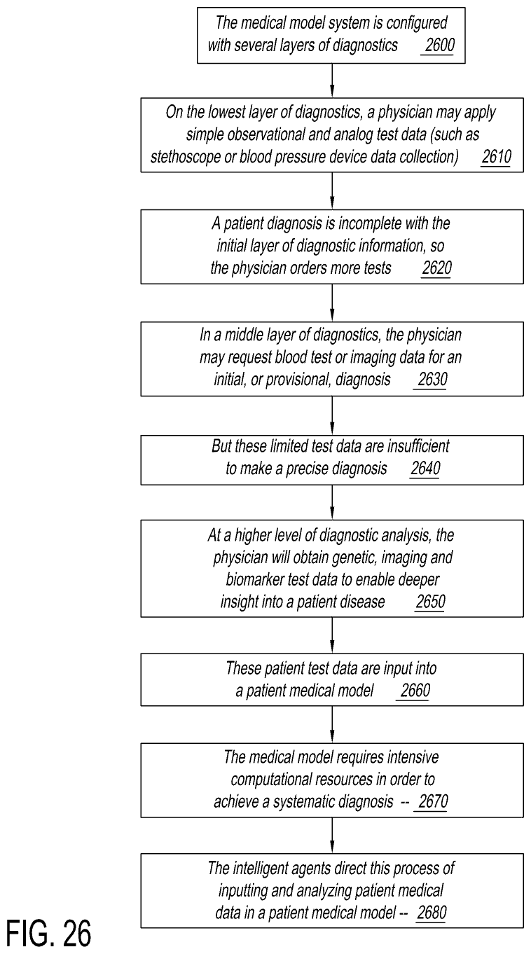

FIG. 26 is a flow chart showing multiple layers of diagnostic analysis in a medical model. The medical model system is configured with several layers of diagnostics (2600). On the lowest layer of diagnostics, a physician may apply simple observational ana analog test data (such as stethoscope or blood pressure device) data collection (2610). A patient diagnosis is incomplete with the initial layer of diagnostic information, so the physician orders more tests (2620). In a middle layer of diagnostics, the physician may request blood test or imaging data for an initial, or provisional, diagnosis (2630) but these limited test data are insufficient to make a precise diagnosis (2640). At a higher level of diagnostic analysis, the physician will obtain genetic, imaging and biomarker test data to enable deeper insight into a patient disease (2650), which patient test data are input into a patient medical model (2660). The medical model requires intensive computational resources in order to achieve a systematic diagnosis (2670). Intelligent agents direct this process of inputting and analyzing patient medical data in a patient medical model (2680).

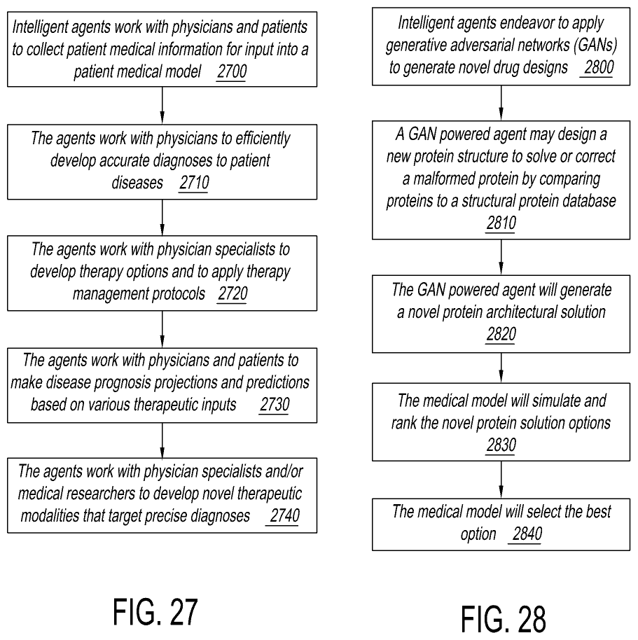

FIG. 27 is a flow chart showing the process of applying intelligent agents to build a medical model. Intelligent agents work with physicians and patients to collect patient medical information for input into a patient medical model (2700) and the agents work with physicians to efficiently develop accurate diagnoses to patient diseases (2710). The agents work with physician specialists to develop therapy options and to apply therapy management protocols (2720). The agents work with physicians and patients to make disease prognosis projections and predictions based on various therapeutic inputs (2730) and the agents work with physician specialists and/or medical researchers to develop novel therapeutic modalities that target precise diagnoses (2740).

FIG. 28 is a flow chart showing application of generative adversarial networks to generate novel drugs. Intelligent agents endeavor to apply generative adversarial networks (GANs) to generate novel drug designs (2800). A GAN powered agent may design a new protein structure to solve or correct a malformed protein by comparing proteins to a structural protein database (2810) and the GAN powered agent will generate a novel protein architectural solution (2820). The medical model will simulate and rank the novel protein solution options (2830) and the model will select the best option (2840).

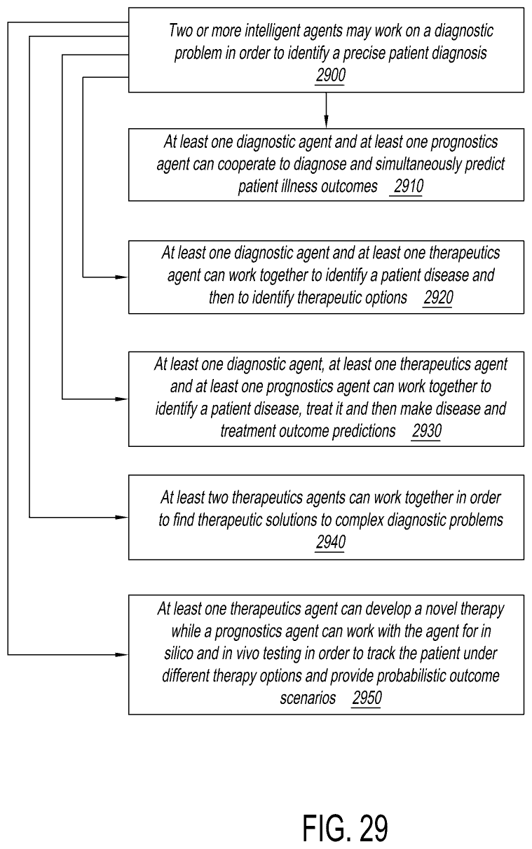

FIG. 29 is a flow chart showing the process of applying two or more intelligent agents for diagnostic modeling. Two or more intelligent agents may work on a diagnostic problem in order to identify a precise patient diagnosis (2900). At least one diagnostic agent and at least one prognostics agent can cooperate to diagnose and simultaneously predict illness outcomes (2910). At least one diagnostic agent and at least one therapeutics agent can work together to identify a patient disease and then to identify therapeutic options (2920). At least one diagnostic agent, at least one therapeutics agent and at least one prognostics agent can work together to identify a patient disease, treat it and then make disease and treatment outcome predictions (2930). At least two therapeutics agents can work together in order to find therapeutic solutions to complex diagnostic problems (2940). At least one therapeutics agent can develop a novel therapy while a prognostics agent can work with the agent for in silico and in vivo testing in order to track the patient under different therapy options and provide probabilistic outcome scenarios (2950).

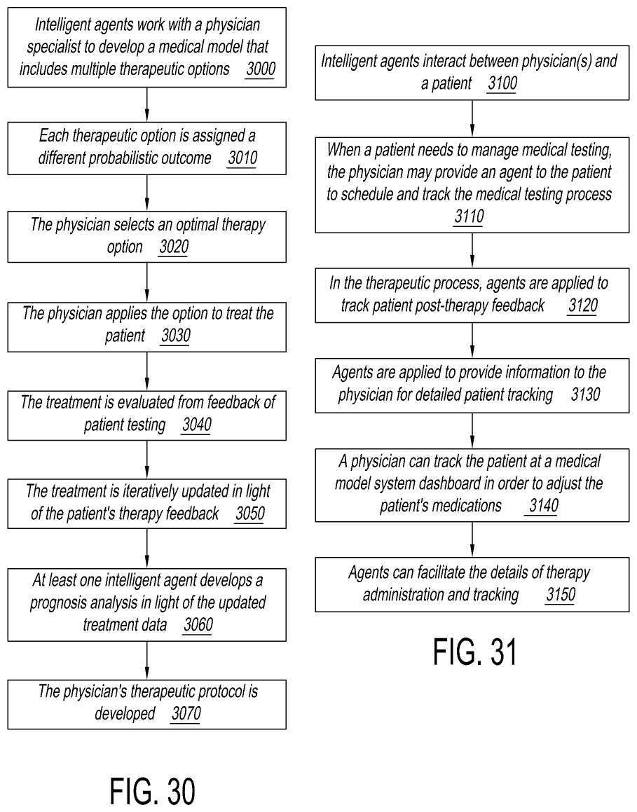

FIG. 30 is a flow chart showing application of intelligent agents to develop a medical model for therapeutic options. Intelligent agents work with a physician specialist to develop a medical model that includes multiple therapeutic options (3000), with each therapeutic option assigned a different probabilistic outcome (3010). The physician selects an optimal therapy option (3020) and applies the option to treat the patient (3030). The treatment is evaluated from feedback of patient testing (3040) and the treatment is iteratively updated in light of the patient's therapy feedback (3050). At least one intelligent agent develops a prognosis analysis in light of the updated treatment data (3060) and the physician's therapeutic protocol is developed (3070).

FIG. 31 is a flow chart showing application of an intelligent agent to patient testing and therapy tracking. Intelligent agents interact between physician(s) and a patient (3100). When a patient needs to manage medical testing, the physician may provide an agent to the patient to schedule and track the medical testing process (3110). In the therapeutic process, agents are applied to track patient post-therapy feedback (3120) and agents are applied to provide information to the physician for detailed patient tracking (3130). A physician can track the patient at a medical model dashboard in order to adjust the patient's medications (3140) and agents can facilitate the details of therapy administration and tracking (3150).

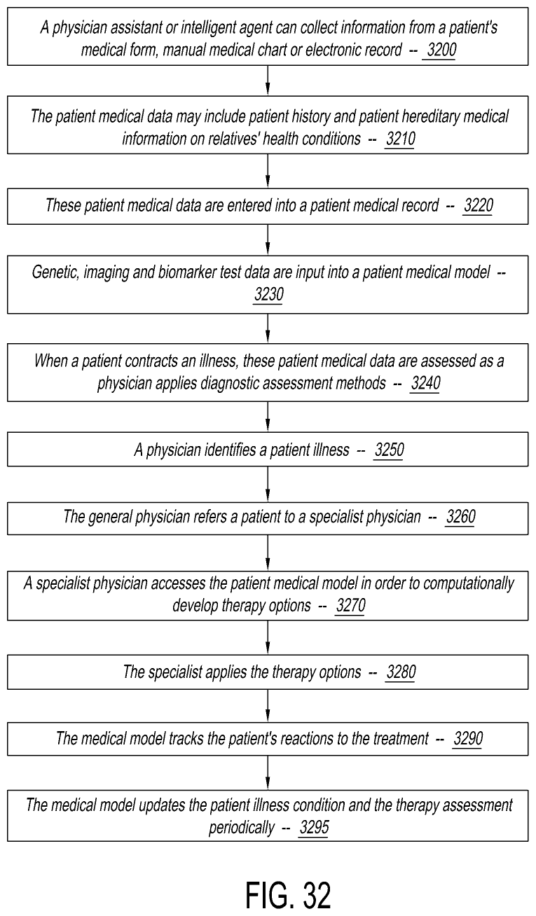

FIG. 32 is a flow chart showing the process of applying an intelligent agent to collect patient data for a diagnostic assessment and therapeutic option assessment. A physician assistant or intelligent agent can collect information from a patient's medical form, manual medical chart or electronic record (3200). The patient medical data may include patient history and patient hereditary medical information on relatives'health conditions (3210). These medical data are entered into a patient medical record (3220). Genetic, imaging and biomarker test data are input into a patient medical model (3230). When a patient contracts an illness, these patient medical data are assessed as a physician applies diagnostic assessment methods (3240). A physician identifies a patient illness (3250) and the general physician refers a patient to a specialist physician (3260). A specialist physician will access the patient medical model in order to computationally develop therapy options (3270) and the specialist will apply the therapy options (3280). The medical model will track the patient's reactions to the treatment (3290) and will update the patient illness condition and the therapy assessment periodically (3295).

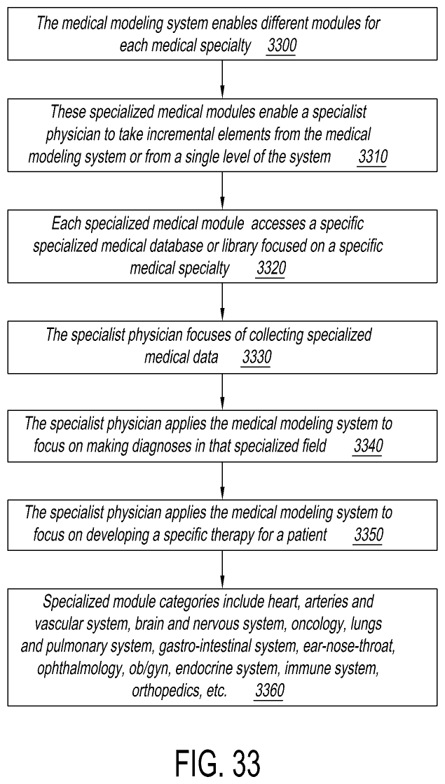

FIG. 33 is a flow chart showing the process of applying specialized medical modules to different medical specialties. The medical modeling system enables different modules for each medical specialty (3300). These specialized medical modules enable a specialist physician to take incremental elements from the medical modeling system or from a single level of the system (3310). Each specialized medical module will access a specific specialized medical database or library focused on a specific medical specialty (3320). The specialist physician focuses on collecting specialized medical data (3330) and applies the medical modeling system to focus on making diagnoses in that specialized field (3340). The specialist physician applies the medical modeling system to focus on developing a specific therapy for a patient (3350). The specialized module categories include heart, arteries and vascular system, brain and nervous system, oncology, lungs and pulmonary system, gastro-intestinal system, ear-nose-throat, ophthalmology, ob/gyn, endocrine system, immune system, orthopedics, etc. (3360).

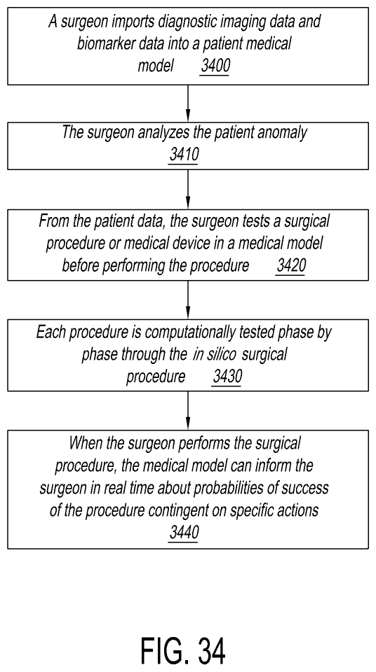

FIG. 34 is a flow chart showing the process of a surgeon applying a medical model to analyze a patient anomaly prior to a surgical procedure. A surgeon imports diagnostic imaging data and biomarker data into a patient medical model (3400). The surgeon analyzes the patient anomaly (3410). From the patient data, the surgeon tests a surgical procedure or medical device in a medical model before performing the procedure (3420). Each procedure is computationally tested phase by phase through the in silico surgical procedure (3430). When the surgeon performs the surgical procedure, the medical model can inform the surgeon in real time about probabilities of success of the procedure contingent on specific actions (3440).

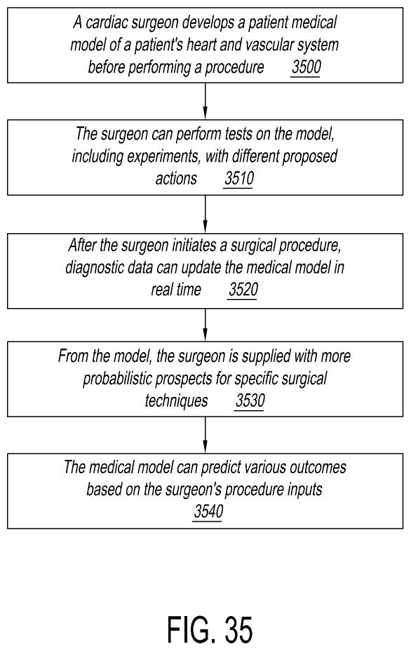

FIG. 35 is a flow chart showing the process of a cardiac surgeon applying a medical model to perform a medical procedure. A cardiac surgeon develops a patient medical model of a patient's heart and vascular system before performing a procedure (3500). The surgeon can perform tests on the model, including experiments, with different proposed actions (3510). After the surgeon initiates a surgical procedure, diagnostic data can update the medical model in real time (3520). From the model, the surgeon is supplied with more probabilistic prospects for specific surgical techniques (3530). The medical model can predict various outcomes based on the surgeon's procedure inputs (3540).

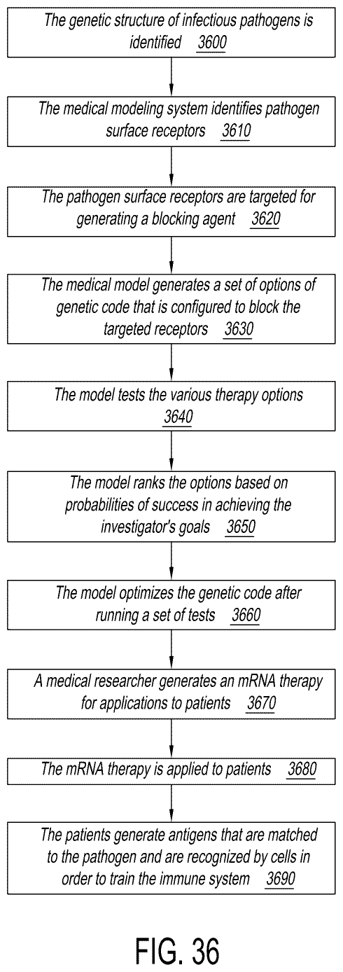

FIG. 36 is a flow chart showing the process of applying a medical model to analyze a pathogen and generate prospective therapies. After the genetic structure of infectious pathogens is identified (3600), the medical modeling system identifies pathogen surface receptors (3610). The pathogen surface receptors are targeted for generating a blocking agent (3620) and the model generates a set of options of genetic code that is configured to block the targeted receptors (3630). The model tests the various therapy options (3640) and ranks the options based on probabilities of success in achieving the investigator's goals (3650). The model optimizes the genetic code after running a set of tests (3660). A medical researcher generates an mRNA therapy for application to patients (3670) and the mRNA therapy is applied to patients (3680). The patients generate antigens that are matched to the pathogen and are recognized by cells in order to train the immune system (3690).

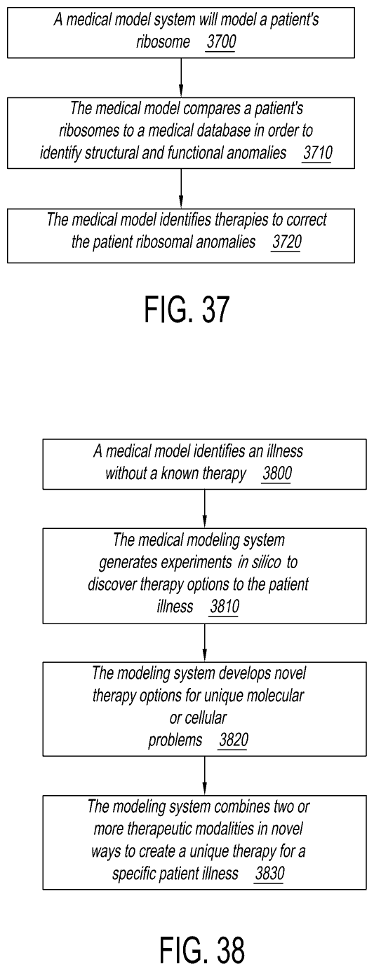

FIG. 37 is a flow chart showing a medical model applied to model ribosomal anomalies and therapies. A medical model system will model a patient's ribosome (3700), the model compares a patient's ribosomes to a medical database in order to identify structural and functional anomalies (3710) and the model identifies therapies to correct the patient ribosomal anomalies (3720). A physician applies gene editing, cellular reprogramming, protein replacement or mRNA therapies to correct the patient ribosomal anomalies (3730).

FIG. 38 is a flow chart showing the process of generating in silico experiments in a medical model to identify novel therapies. A medical model identifies an illness without a known therapy (3800) and the medical modeling system generates experiments in silico to discover therapy options to the patient illness (3810). The modeling system develops novel therapy options for unique molecular or cellular problems (3820) and the modeling system combines two or more therapeutic modalities in novel ways to create a unique therapy for a specific patient illness (3830).

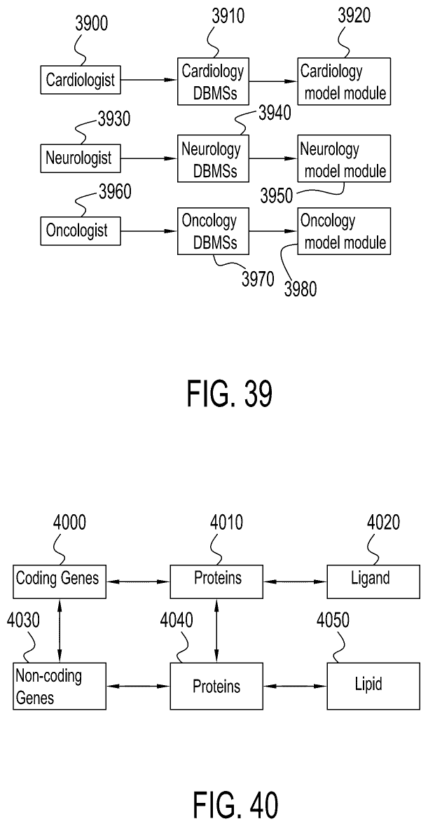

FIG. 39 is a drawing of specialized medical modules derived from specialized medical databases. A cardiologist (3900) accesses a cardiology database (3910) and develops a cardiology model module (3920). A neurologist (3930) accesses a neurology database (3940) and develops a neurology model module (3950). An oncologist (3960) accesses an oncology database (3970) and develops an oncology model module (3980).

FIG. 40 is a diagram of medical models and simulations of functional molecular interactions. Referring to level 5, which specifies models and simulations of functional molecular interactions, the figure show coding genes (4000) interacting with non-coding genes (4030) and proteins (4010). Proteins (4010) and shown interacting with proteins (4040), ligands (4020) and lipids (4050).

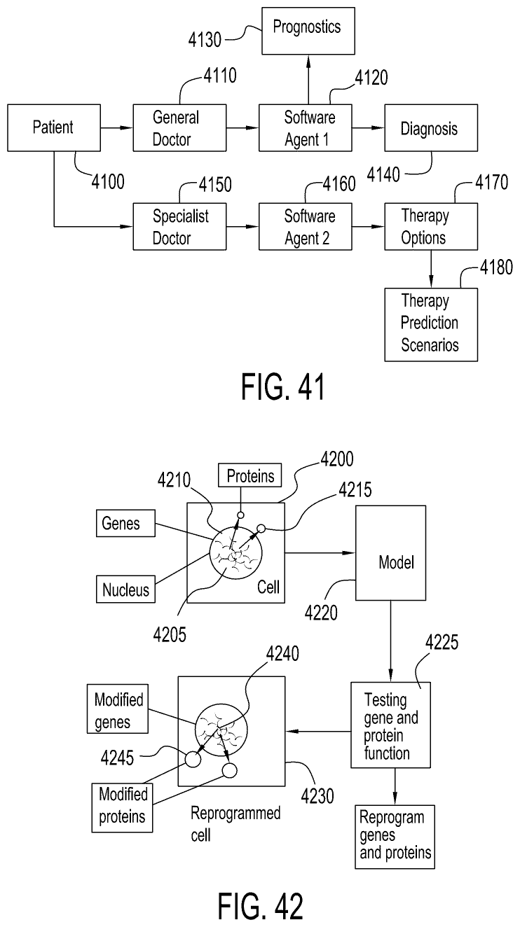

FIG. 41 is a diagram of software agents applied to diagnostics, prognostics and therapeutics in a medical modeling system. A patient (4100) interacts with a general practitioner (4110) and a specialist doctor (4150). Software agent 1 (4120) interfaces with a general practitioner (4110) in order to develop a diagnosis (4140) and a prognosis (4130) of a patient illness. Software agent 2 (4160) interfaces with a specialist doctor (4150) in order to develop therapy options (4170) and therapy prediction scenarios (4180).

FIG. 42 is a diagram of a medical model of a molecular and cellular anomaly and the testing and reprogramming of gene and protein functions. A cell (4200) is shown with a nucleus (4205), genes (4210) and proteins (4215). The cell and molecular phenomena are modeled (4220). The model tests the gene and protein functions and reprograms the genes and proteins in silico (4225). From the model, the cell (4230) is reprogrammed, with modified genes (4235) and modified proteins (4245).

Although the present invention has been described in relation to particular embodiments thereof, many other variations and other uses will be apparent to those skilled in the art. It is preferred, therefore, that the present invention be limited not by the specific disclosure herein, but only by the gist and scope of the disclosure.

I claim:

1. A system for medical modeling, the system comprising:
a computer modeling system consisting of least one of logic circuit, including at least one GPU, CPU, FPGA, neuromorphic or ASIC logic circuit and at least one of DRAM or SRAM memory circuit;
a database management system to store and access data;
computer modeling software;
at least one of a set of modular levels configured to diagnose, predict or treat a patient's medical condition, the system including:
Level 1 [General Patient Model];
Level 2 [Bioinformatics Analysis];
Level 3 [Molecular and Cellular Description];
Level 4 [Structural Genetic Combination Pathology Identification];
Level 5 [Functional Molecular and Cellular Pathology Diagnosis];
Level 6 [Diagnostic Prognosis Simulation];
Level 7 [General Therapy Solutions];
Level 8 [Unique Therapy Solution Genesis];
Level 9 [Therapy Option Testing and Simulations];
Level 10 [Therapy Prediction Scenarios];
Level 11 [Unified Patient Model]; and
Level 12 [Human Population Model].
